(19) United States
(12) Patent Application Publication
(10) Pub. No.: US 2026/0115694 A1
(43) Pub. Date: Apr. 30, 2026

(54) Single-atom Nanozymes, Preparation and Applications thereof

(71) Applicant: City University of Hong Kong, Kowloon (HK)

(72) Inventors: Chun-Sing Lee, Kowloon (HK); Weifeng Yao, Shanghai (CN); Qi Zhao, Kowloon (HK)

(21) Appl. No.: 19/003,238

(22) Filed: Dec. 27, 2024

Related U.S. Application Data
(60) Provisional application No. 63/615,019, filed on Dec. 27, 2023.

(51) Int. Cl. B01J 23/02; A61K 33/06; A61K 39/00; A61P 35/00; B01J 27/20; B01J 29/04; B01J 35/45; B01J 37/04; B01J 37/08

(52) U.S. Cl. CPC B01J 23/02; A61K 33/06; A61K 39/00; A61P 35/00; B01J 27/20; B01J 29/04; B01J 35/45; B01J 37/04; B01J 37/082; A61K 2039/55505

(57) ABSTRACT

A single atom nanozyme includes an alkaline earth metal single atom loaded on a carbon material, in which the alkaline earth metal is calcium, magnesium, or barium, and the carbon material is nitrogen-doped and can be selected from imidazolate framework (ZIF), carbon fiber, carbon nanotube, graphene, carbon black, and reduced graphene oxide. Also provided is a method for preparing the single atom nanozymes, and a method of tumor catalytic therapy using the single atom nanozymes.

### SEQUENCE LISTING

The Sequence Listing file entitled “sequencelisting” having a size of 9,437 bytes and a creation date of Dec. 27, 2025, that was filed with the patent application is incorporated herein by reference in its entirety.

### FIELD OF THE INVENTION

The invention relates to the technical field of nanozymes, in particular single atom nanozymes.

### BACKGROUND

Nanozymes are a type of artificial enzyme that are designed and engineered at the nanoscale level. They mimic the functions of natural enzymes and can catalyze specific chemical reactions. Nanozymes have unique properties such as high stability, reusability, and enhanced catalytic activity, making them promise for various applications in fields like medicine, energy, and environmental remediation. They offer potential advantages over natural enzymes, including easier synthesis, modification, and control over their properties.

Nanozymes exhibit various bioactivities due to their unique properties and catalytic capabilities.

Some of the bioactivities of nanozymes include biocatalysis. Nanozymes can catalyze specific biochemical reactions, similar to natural enzymes. They can accelerate chemical reactions, convert substrates into desired products, and facilitate complex biological processes. In drug delivery, nanozymes can be used as carriers for targeted drug delivery. They can encapsulate therapeutic agents and release them at specific sites in the body, improving drug efficacy and reducing side effects. With biosensing, nanozymes can be utilized in biosensors for detecting and quantifying biomolecules or analytes. They can catalyze reactions that produce detectable signals, enabling sensitive and selective detection of various substances. In bioimaging, nanozymes can be employed in bioimaging techniques to visualize specific biological processes or structures. They can generate signals or contrast agents that enhance imaging resolution and sensitivity. For tissue engineering, nanozymes can play a role in tissue engineering by promoting cell growth, differentiation, and tissue regeneration. They can create favorable microenvironments for cell adhesion, proliferation, and tissue formation.

Developing nanozymes comes with several challenges and difficulties. Designing and synthesizing nanozymes with desired catalytic properties can be complex. It requires a deep understanding of the target reaction, substrate specificity, and the desired catalytic mechanism. Achieving optimal catalytic activity and stability while maintaining biocompatibility can be challenging. Ensuring the scalability and reproducibility of nanozyme synthesis is crucial for their practical applications. It can be difficult to maintain consistent catalytic performance across different batches or scales of production. Standardization of synthesis methods and quality control measures are necessary to address this challenge. Nanozymes need to exhibit long-term stability and durability to be effective in various applications. They should be resistant to degradation, maintain their catalytic activity under different environmental conditions, and withstand potential interactions with biological systems. Nanozymes intended for biomedical applications must be biocompatible and non-toxic to ensure their safety. Understanding the potential interactions between nanozymes and biological systems, including cells and tissues, is crucial to minimize any adverse effects. Achieving high target specificity and selectivity is essential for nanozymes to perform their intended functions accurately. Ensuring that nanozymes only catalyze the desired reactions and do not interfere with other biological processes can be challenging. The development and commercialization of nanozymes may require compliance with regulatory frameworks. Meeting the necessary safety and efficacy standards, as well as navigating the regulatory landscape, can be time-consuming and resource intensive.

Addressing these difficulties requires interdisciplinary collaboration, advanced characterization techniques, and continuous research efforts to optimize the design, synthesis, and application of nanozymes.

The invention seeks to provide new or otherwise improved nanozymes.

### SUMMARY OF THE INVENTION

An embodiment herein relates to a single atom nanozyme comprising an alkaline earth metal single atom loaded on a carbon material, wherein the alkaline earth metal is selected from the group consisting of calcium, magnesium, barium, and a combination thereof, wherein the carbon material is nitrogen-doped and selected from the group consisting of zeolitic imidazolate framework (ZIF), ZIF-8, ZIF-67, carbon fiber, carbon nanotube, graphene, carbon black, reduced graphene oxide, and a combination thereof.

Without intending to be bound by theory, it is believed that the nanozymes according to this invention can act as immunoadjuvants, stimulating cytokine production and promoting the polarization of tumor-associated macrophages to reverse the immunosuppressive microenvironment. These suggest that the nanozymes according to this invention hold promise as artificial enzymes capable of addressing the challenges of Glioblastoma and other immunosuppressive tumors, offering an innovative approach to treatment.

An embodiment herein relates to a method for preparing the single atom nanozymes herein, comprising the steps of:
(A) providing a carbon material selected from the group consisting of zeolitic imidazolate framework (ZIF), carbon fiber, carbon nanotube, graphene, carbon black, reduced graphene oxide, and a combination thereof, and
(B) mixing the nitrogen-doped carbon material with an alkaline earth metal source comprising an alkaline earth metal selected from the group consisting of calcium, magnesium, barium, and a combination thereof and stirring at room temperature to provide a precursor mixture;
(C) subjecting the precursor mixture to a pyrolysis process to provide the single atom nanozymes.

Without intending to be bound by theory, it is believed that the preparation method according to this invention can provide nanozymes with proper particle sizes. For example, the nanozymes according to this application may have a particle size in range of about 50 nm to about 100 nm, which is smaller than those of nanoparticles previously reported. For example, the nanoparticles previously reported by the inventor (U.S. Ser. No. 18/825,151 by Lee et al. to City University of Hong Kong) normally has a particle size in range of about 200 nm to about 300 nm. Without intending to be bound by theory, it is believed that such small particle sizes are beneficial for delivering the nanozymes across the blood-brain barrier (BBB).

An embodiment herein relates to a method of tumor catalytic therapy comprising administrating a nanozyme comprising a single-atom alkaline earth metal loaded on a nitrogen-doped carbon material to a subject in need thereof, wherein the alkaline earth metal is selected from the group consisting of calcium, magnesium, barium, and a combination thereof, wherein the carbon material is selected from the group consisting of zeolitic imidazolate framework (ZIF), carbon fiber, carbon nanotube, graphene, carbon black, reduced graphene oxide, and a combination thereof.

Without intending to be bound by theory, it is believed that the method of tumor catalytic therapy according to this invention can provide efficient catalytic therapy of gliomas and other refractory tumors, with minimal side effects.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will now be more particularly described, by way of example only, with reference to the accompanying drawings, in which:

FIG. 1 is a diagram showing the preparation of Ca-SAzyme and schematic representation of its antitumor function;

FIG. 2A shows a HR-TEM image of ZIF-8;

FIG. 2B shows a SEM image of ZIF-8;

FIG. 2C shows a HR-TEM image of Ca/ZIF-8;

FIG. 2D shows XRD patterns of NC and Ca-SAzyme according to an embodiment of this application;

FIG. 3A shows a HR-TEM image of Ca-SAzyme according to an embodiment of this application;

FIG. 3B shows a HAADF-STEM image of Ca-SAzyme and the corresponding elemental mapping spectroscopy (EDS) for carbon (C), nitrogen (N), and calcium (Ca) in Ca-SAzyme according to an embodiment of this application;

FIG. 3C shows a HAADF-STEM image of Ca-SAzyme according to an embodiment of this application;

FIG. 3D shows electron energy loss spectroscopy (EELS) spectra for atomic sites of Ca-SAzyme according to an embodiment of this application;

FIG. 3E shows nitrogen-sorption data for the Ca-SAzyme according to an embodiment of this application;

FIG. 3F shows the pore size distribution for the Ca-SAzyme according to an embodiment of this application;

FIG. 3G shows the transmission spectra of NC and the Ca-SAzyme according to an embodiment of this application;

FIG. 3H shows the X-ray photoelectron (XPS) survey spectra of the Ca-SAzyme Ca-SAzyme according to an embodiment of this application;

FIG. 3I shows N Is high-resolution XPS spectra of Ca-SAzyme according to an embodiment of this application;

FIG. 3J shows Ca 2p high-resolution XPS spectra of Ca-SAzyme according to an embodiment of this application;

FIG. 3K shows Ca L-edge X-ray absorption near-edge structure of Ca-SAzyme according to an embodiment of this application;

FIG. 3L shows C K-edge X-ray absorption near-edge structure of Ca-SAzyme according to an embodiment of this application;

FIG. 3M shows N K-edge X-ray absorption near-edge structure of Ca-SAzyme according to an embodiment of this application;

FIG. 4A shows Ca K-edge of XANES curves of CaO and Ca-SAzyme according to an embodiment of this application;

FIG. 4B shows Ca K-edge FT-EXAFS spectra of Ca-SAzyme and Cao at R-space;

FIG. 4C shows Ca K-edge FT-EXAFS spectra of Ca-SAzyme and Cao at K-space;

FIG. 4D is a diagram showing the atomic structural simulations of Ca-SAzyme, in which the insets are the schematic models of Ca-SAzyme (Atom colors: green, Ca; dark gray, C; blue, N);

FIG. 4E shows the WT analysis of CaO;

FIG. 4F shows the WT analysis of Ca-SAzyme according to an embodiment of this application;

FIG. 5A is a plot showing different Ca-SAzyme number of ratios of the TMB colorimetric reaction catalyzed by Ca-SAzyme according to an embodiment of this application;

FIG. 5B is a plot showing peroxidase-like activity of the Ca-SAzyme at different TMB concentrations;

FIG. 5C is a plot showing peroxidase-like activity of the Ca-SAzyme at different H₂O₂ concentrations;

FIG. 5D is a plot showing reaction-time curves of the TMB colorimetric reaction catalyzed by Ca-SAzyme;

FIG. 5E is a plot showing reaction-time curves of the H₂O₂ colorimetric reaction catalyzed;

FIG. 5F is a plot showing the specific activities (U mg⁻¹) of Ca-SAzyme according to an embodiment of this application;

FIG. 5G is a plot showing the comparison of peroxidase-like activity among blank, Ca-SAzyme, ZIF-8, and ZIF-8 (Ca²⁺) by using TMB liquid substrate system;

FIG. 5H is a plot showing the comparison of peroxidase-like activity among Ca-SAzyme, graphitic carbon nitride and graphene oxide by using TMB liquid substrate system;

FIG. 5I is a plot showing PH-dependent peroxidase-like activities of Ca-SAzyme according to an embodiment of this application;

FIG. 5J is a plot showing temperature-dependent peroxidase-like activities of Ca-SAzyme according to an embodiment of this application;

FIG. 5K is a plot showing the comparison of PL intensity among Ca-SAzyme according to an embodiment of this application;

FIG. 6A shows the confocal microscopic images for cells treated with Ca-SAzyme (0, 20, 50, 200 μg mL⁻¹ in diagram i, ii, iii, iv), H₂O₂ (100 μM) and their combination, using DCFH-DA cell-staining, Scale bars: 100 μm;

FIG. 6B shows the confocal microscopic images for cells treated with Ca-SAzyme (0, 20, 50, 200 μg mL⁻¹ in diagram i, ii, iii, iv), H₂O₂ (100 μM) and their combination, using Calcein-AM/PI cell-staining;

FIG. 6C are diagrams showing Annexin V/PI assay of GBM 21 cells treated with Ca-SAzymes (0, 20, 50, 200 μg mL⁻¹ in diagram i, ii, iii, iv) for 24 h and without H₂O₂;

FIG. 6D is a plot showing GSEA analysis feedback results that Ca-SAzymes according to an embodiment of this application give rise to reactive oxygen species: Apoptosis;

FIG. 6E is a plot showing GSEA analysis feedback results that Ca-SAzymes according to an embodiment of this application give rise to reactive oxygen species: Inflammatory;

FIG. 6F is a plotting showing GSEA analysis feedback results that Ca-SAzymes according to an embodiment of this application give rise to reactive oxygen species: Enrichment;

FIG. 7A is a chart showing cytotoxicity of Ca-SAzymes to astrocytes NHA in weakly acidic environments (pH=6),

FIG. 7B is a chart showing cytotoxicity of Ca-SAzymes to astrocytes NHA in neutral environments (pH=7.4);

FIG. 7C is a chart showing in vitro killing capacity of Ca-SAzymes to breast tumor cell MDA231;

FIG. 7D is chart showing in vitro killing capacity of Ca-SAzymes to lung tumor cell NCl-H520;

FIG. 7E is a chart showing cytotoxicity of ZIF-8 to GSC 21 (pH of 7.4);

FIG. 7F is a chart showing cytotoxicity of ZIF-8 to GSC 21 (pH=6);

FIG. 8 shows western blot analysis of Calpain-1 and Clv-caspase 3 in GBM 21 cells after varied treatments;

FIG. 9 shows enriched chord diagram of the KEGG pathways;

FIG. 10 shows Boxplots of mesenchymal transition activity across 33 cancer types, as inferred using ssGSEA based on EMTCGs signature;

FIG. 11A is a chart showing results of GBM 21 cells viability assays of Ca-SAzymes without H₂O₂ under neutral culture media (pH=7.4);

FIG. 11B is a chart showing results of GBM 22 cell viability assays of Ca-SAzymes without H₂O₂ under neutral culture media (pH=7.4);

FIG. 11C is a chart showing results of viability assays of the cells pre-incubated with Ferrostatin-1 (200 μM), and 3-MA (200 μM) followed by Ca-SAzymes treatment under neutral culture media, Condition: Ca-SAzymes: 200 μg mL⁻¹, pH=7.4. Values are presented as means±s.d. (experiments were performed in quadruplicates). *P<0.05, **P<0.01, ***P<0.001;

FIG. 12 is a diagram showing cytotoxicity of Ca-SAzymes in different GBM microenvironment (20 C=20 μg/ml Ca-SAzymes, 50 C=50 μg/ml Ca-SAzymes, 200 C=200 μg/ml Ca-SAzymes);

FIG. 13A is a diagram showing PCA of ROS metabolic capacity of Ca-SAzymes in different tumours and tumours in different locations;

FIG. 13B shows volcano map revealing the downregulated or up regulated genes of Ca-SAzymes group, as compared to the control group;

FIG. 13C shows the circle diagram of GO and KEGG analyses according to an embodiment of this application;

FIG. 13D shows the heat map of certain interesting genes in GO analyses according to an embodiment of this application;

FIG. 13E are diagrams showing differential expression of cancer-related genes according to an embodiment of this application;

FIG. 13F is a chart showing the quantitative analysis of TNF-α and IFN-γ mRNA expressions based on PCR results;

FIG. 13G is a chart showing corresponding quantitative analysis of NOS2 and IL6 mRNA expressions based on PCR results;

FIG. 13H is an image showing western blot analysis of MRC1 and NOS2 after varied treatments;

FIG. 14A is a schematic diagram illustrating the timeline for antitumour treatment of Ca-SAzymes;

FIG. 14B shows fluorescence imaging of glioblastoma-bearing nude mice scanned after injection of Ca-SAzymes in situ;

FIG. 14C is a plot showing the survival rate of glioblastoma-bearing nude mice under different treatments (log-rank test, n=5);

FIG. 14D is a plot showing the time-dependent body weights of glioblastoma-bearing nude mice under different treatments (log-rank test, n=5);

FIG. 14E shows hematoxylin and eosin staining and the quantification of the relative maximum cross-sectional area of orthotopic GBM tumours; Scale bar: 500 μm;

FIG. 14F is a chart showing M1/M2 of glioblastoma-bearing C57BL6 mice under different treatments, Error bars indicate the mean means±s.d.; *p<0.05, **p<0.01, ***p<0.001;

FIG. 14G is a chart showing CD4+/Treg of glioblastoma-bearing C57BL6 mice under different treatments, Error bars indicate the mean means±s.d.; *p<0.05, **p<0.01, ***p<0.001;

FIG. 14H is a chart showing CD8+/Treg of glioblastoma-bearing C57BL6 mice under different treatments, Error bars indicate the mean means±s.d.; *p<0.05, **p<0.01, ***p<0.001;

FIG. 14I shows hematoxylin and eosin staining of multiple organs collected from nude mice at the end of treatment. Scale bar: 100 μm;

FIG. 15A is a diagram showing the obtained OH+OH adsorption structure after HOOH adsorption with the key distances marked in Å, *presents the absorbers absorbed on the surface;

FIG. 15B is a diagram showing the differential charge density distributions for CaN₃ doped graphene and OH+OH adsorption (blue: accepting electrons, and red: donating electrons);

FIG. 15C shows the energy profile diagrams of *OH+*OH+*H→*OH+*H₂O reactions with the structures of transition states (TS) located next to the curve;

FIG. 15D shows the energy profile diagrams of *OH+*H→*H₂O reactions with the structures of transition states (TS) located next to the curve;

FIG. 15E is a diagram showing optimized structure for CaN₃ doped graphene;

FIG. 15F is an energy diagram for HOOH decomposition on the CaN₃ doped graphene;

FIG. 16 are images showing TEM analysis of mitochondria, in which red arrows in the pictures indicate the positions of mitochondria, Scale bar: 500 nm; and

FIG. 17A is a chart showing TNF-α levels (pg/ml) in GBM culture after different treatments, Ca-SAzyme: 0, 20, 50, 200 μg mL⁻¹;

FIG. 17B is a chart showing IFN-γ levels (pg/ml) in GBM culture after different treatments, Ca-SAzyme: 0, 20, 50, 200 μg mL⁻¹; and

FIG. 17C is a chart showing IL-Iβ levels (pg/ml) in GBM culture after different treatments, Ca-SAzyme: 0, 20, 50, 200 μg mL⁻¹.

The figures herein are for illustrative purposes only and are not necessarily drawn to scale.

### DESCRIPTION OF THE PREFERRED EMBODIMENTS

It is understood that unless otherwise specifically noted, the materials compounds, chemicals, etc. described herein are typically commodity items and/or industry-standard items available from a variety of suppliers worldwide.

### Definitions

As used herein, “nanozyme(s)” refers to nanomaterials with intrinsic enzyme-like activity that catalyze the conversion of substrates by following the same kinetics and mechanisms as natural enzymes under physiological condition.

As used herein, “alkaline earth metal single atom nanozymes” refer to nanoscale particles or clusters consisting of single atoms of alkaline earth metals, such as strontium (Sr), radium (Ra), calcium, magnesium, and barium.

As used herein, “glioblastoma”, “GBM” refers to glioblastoma multiforme, the most malignant type of glioma.

As used herein, “therapeutically effective amount” refers to is any amount of the drug that, when used alone or in combination with another therapeutic agent, protects a subject against the onset of a disease or facilitates the regression of a disease. This regression is typically characterized by a reduction in the severity of disease symptoms, an increase in the frequency and duration of symptom-free periods, or the prevention of impairment or disability caused by the disease. The ability of a therapeutic agent to promote disease regression can be evaluated using a variety of methods known to the skilled practitioner, such as in clinical trials involving human subjects, in animal model systems that are predictive of efficacy in humans, or by evaluating the agent's activity in in vitro assays.

As used herein, “refractory microenvironmental immunosuppressive tumor” refers to tumors that have the ability to regulate the phenotype of peritumoral immune cells, especially the transformation of pro-inflammatory M1 macrophages into anti-inflammatory M2 macrophages, including but not limited to, breast cancer, glioma, and some digestive tract tumors.

“Tumor immune microenvironment (TIME)” as used herein is organized by a variety of immune cells and stromal cells, whose heterogeneity is a major contributing factor to tumor metastasis, recurrence, and drug resistance.

“Immunosuppressive microenvironment” is the microenvironment that tumor “produces” to inhibit immune effector cells and promote their own growth.

An embodiment herein relates to a single atom nanozyme comprising an alkaline earth metal single atom loaded on a carbon material.

Natural enzymes have disadvantages such as high cost, poor stability, low yield, difficult storage and short half-life, which seriously limit the practical application of enzyme therapy as a catalytic therapy. Therefore, it is believed that the emergence of nanozymes as a tumor catalytic therapy is of great significance. Without intending to be bound by theory, it is believed that the nanozymes according to this invention can act as immunoadjuvants, stimulating cytokine production and promoting the polarization of tumor-associated macrophages to reverse the immunosuppressive microenvironment.

In some embodiments, the single atom nanozyme comprises the alkaline earth metal selected from calcium, magnesium, barium, and a combination thereof. In some embodiments, the alkaline earth metal is calcium. Without intending to be bound by theory, it is believed that metals such as Fe, Zn, Pt, Cu, and Co have been explored in SAzymes, but further improvements in economic viability and biocompatibility are needed. Calcium (Ca), the fifth most abundant element in Earth's crust, is one of the most economical and biocompatible metals, making it an attractive candidate for catalytic applications. In addition, it is believed that single-atom alkaline earth metals have not been explored in nanozymes, and the mechanisms by which single-atom doping influences catalytic performance remain unclear in prior art.

In some embodiments, the single atom nanozyme comprises the carbon material that is nitrogen-doped and selected from zeolitic imidazolate framework (ZIF), carbon fiber, carbon nanotube, graphene, carbon black, reduced graphene oxide, and a combination thereof. In some embodiments, the single atom nanozyme comprises zeolitic imidazolate framework selected from ZIF-8, ZIF-67, and a combination thereof. More particularly, the zeolitic imidazolate framework is ZIF-8.

Without intending to be bound by theory, it is believed that Zeolitic Imidazolate Frameworks (ZIFs), particularly ZIF-8 and ZIF-67, are often preferred as precursors for single-atom nanozymes (SAzymes) due to their unique structural and chemical properties, at least for the following reasons:

### 1. Porous Structure for High Surface Area

Advantage: ZIFs have a highly porous framework with a large surface area, enabling better accessibility of reactants to the active sites of the nanozyme.

Benefit for Nanozymes: This enhances catalytic efficiency and activity, making ZIF-derived materials highly effective.

### 2. Uniform Metal Distribution

Advantage: The metal ions (e.g., Zn in ZIF-8 or Co in ZIF-67) are uniformly distributed within the framework.

Benefit for Nanozymes: This uniformity aids in the formation of well-dispersed single-atom catalysts after pyrolysis, which is crucial for maximizing catalytic activity.

### 3. Thermal Stability

Advantage: ZIFs possess high thermal and chemical stability, allowing them to withstand the conditions needed for carbonization.

Benefit for Nanozymes: During the conversion process (e.g., pyrolysis), the framework transforms into a stable carbon structure, embedding the single atoms effectively.

### 4. Tailored Properties Via Composition

ZIF-8 (Zn-based): Tends to form nitrogen-doped carbon frameworks upon pyrolysis, which are beneficial for catalytic activity in oxidative and reductive reactions.

ZIF-67 (Co-based): Generates cobalt-doped carbon with strong activity in redox reactions and high electron conductivity.

### 5. Nitrogen-Rich Framework

Advantage: ZIFs are constructed using imidazolate linkers, which are nitrogen rich.

Benefit for Nanozymes: Upon pyrolysis, the nitrogen atoms form stable metal-N—C (metal-nitrogen-carbon) active sites that are essential for the catalytic performance of nanozymes.

### 6. Versatility and Tunability

Advantage: ZIFs allow for structural and compositional tunability by changing the metal ions or linkers.

Benefit for Nanozymes: This flexibility enables researchers to tailor the properties of the nanozyme for specific catalytic applications, such as cancer treatment, environmental remediation, or energy conversion.

### 7. Cost-Effectiveness and Scalability

Advantage: ZIFs can be synthesized relatively easily and cost-effectively at a large scale.

Benefit for Nanozymes: This makes them practical for industrial applications and large-scale production.

### 8. Precursor for Hierarchical Structures

Advantage: ZIF-derived materials can produce hierarchical porous structures after pyrolysis.

Benefit for Nanozymes: This provides a synergistic effect by combining high surface area, mass transport efficiency, and active site accessibility.

By selecting ZIF-8 or ZIF-67 as a precursor, these combined advantages make them highly effective and versatile materials for creating high-performance single-atom nanozymes.

In an embodiment herein, the single atom nanozyme includes calcium, which mimic the active sites of natural metalloproteases. In an embodiment herein, ZIF-8 or ZIF-67 is used as the carbon material. In some embodiments, the alkaline earth metal is calcium, and the zeolitic imidazolate framework is ZIF-8.

In an embodiment herein, the single atom nanozymes herein have a particle size ranging from about 50 nm to about 100 nm. For example, the single atom nanozymes herein may have a particle size of about 60 nm, about 70 nm, about 80 nm, or about 90 nm. Without intending to be bound by theory, it is believed that nanozymes' small size provides a high surface-area-to-volume ratio, exposing more catalytic active sites. Single-atom nanozymes (SAzymes), in particular, offer exceptional catalytic stability and tunability across a range of reactions. Without intending to be bound theory, it is believed that the nanozymes prepared according to this application are capable of being delivered across BBB and to glioma core, and thus have high activity. It is also believed that the nanozymes prepared using the method herein have more stable catalytic activities and biosafety compared with existing nanozymes.

In an embodiment herein, the single atom nanozymes have a CaN₃ structure.

With reference to the figures, the invention discloses an alkaline earth metal single atom nanozyme material and its application in biomedicine. Alkaline earth metal single atom nanozymes refer to nanoscale particles or clusters consisting of single atoms of alkaline earth metals, such as magnesium (Mg), calcium (Ca), strontium (Sr), barium (Ba), and radium (Ra). These nanozymes exhibit enzyme-like catalytic activity, similar to natural enzymes, due to the unique electronic and structural properties of the single metal atoms. Without intending to be bound by theory, it is believed that the alkaline earth metal single atom nanozyme according to this invention has high catalytic efficiency, stability, and selectivity. It is also believed that the nanozymes according to this invention can be synthesized with precise control over their size, composition, and surface properties by the method described herein, allowing for tailored catalytic performance. Without intending to be bound by theory, it is believed that the nanozymes according to this invention can be easily integrated into different systems, such as nanomedicine platforms, biosensing, cancer therapy, water purification, and energy storage.

An embodiment herein relates to a method for preparing the single atom nanozymes herein, comprising the steps of:
(A) providing a carbon material selected from the group consisting of zeolitic imidazolate framework (ZIF), carbon fiber, carbon nanotube, graphene, carbon black, reduced graphene oxide, and a combination thereof, and
(B) mixing the nitrogen-doped carbon material with an alkaline earth metal source comprising an alkaline earth metal selected from the group consisting of calcium, magnesium, barium, and a combination thereof and stirring at room temperature to provide a precursor mixture;
(C) subjecting the precursor mixture to a pyrolysis process to provide the single atom nanozymes.

During step (B), the alkaline earth metal is absorbed into the solution of the nitrogen-doped carbon material, for example, by using ion exchange procedure. In some examples, alkaline earth metal aqueous solution (for example, CaCl₂), 1 g mL⁻¹, 500 μL) is added into the ZIF solution slowly under stirring. Next, the mixture is vigorous stirred at room temperature so that the salt solution is absorbed completely to form the precursor, which is then subjected to the pyrolysis process under step (C). For example, CaCl₂) is added and absorbed into ZIF-8 solution to form Ca/ZIF-8, which is then pyrolyzed.

During step (C), organic MOF ligands are transformed to an N-doped carbon structure during the pyrolysis process, and the N-rich porous carbon trapped the alkaline earth metal atoms to create the nanozymes.

The invention is related to the method of preparation of alkaline earth metal single-atom nanozymes by the host-guest method. Without intending to be bound by theory, it is believed that the fabricated catalyst demonstrates the specific and efficient peroxidase-like activity. It is also believed that the preparation method according to this invention can synthesize nanozymes with precise control over their size, composition, and surface properties.

The single atom nanozyme material utilizes carbon and calcium sources by the host-guest method. As a specific example, carbon-based materials, such as carbon nanotubes or graphene, and calcium sources are used to create a host-guest system and is synthesized by room temperature stirring and high temperature calcination with calcium single atoms loaded on a nitrogen-doped carbon substrate.

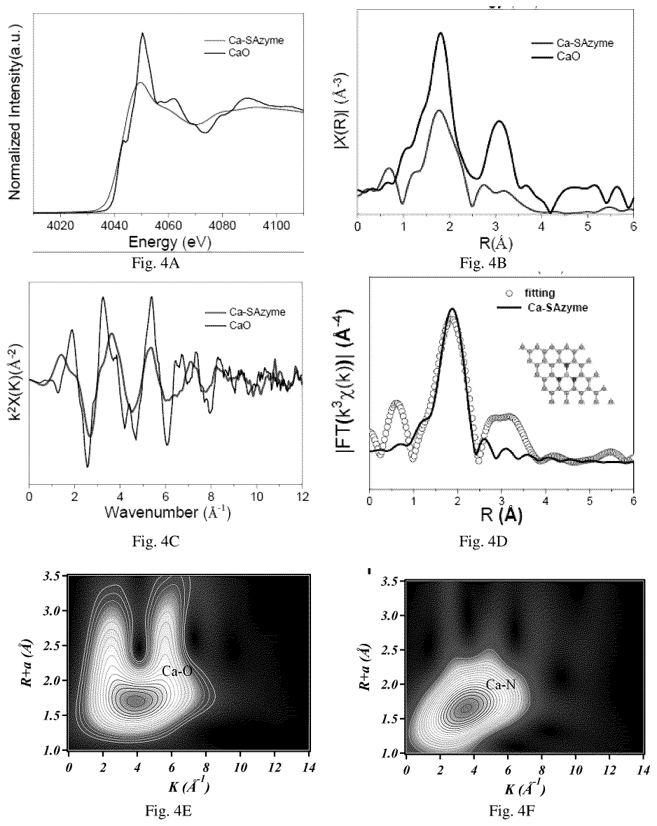

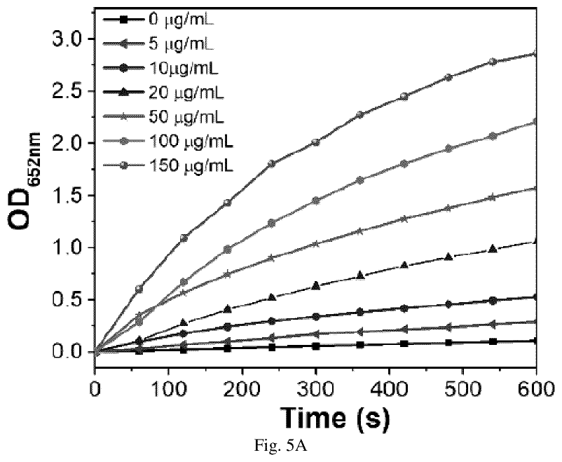

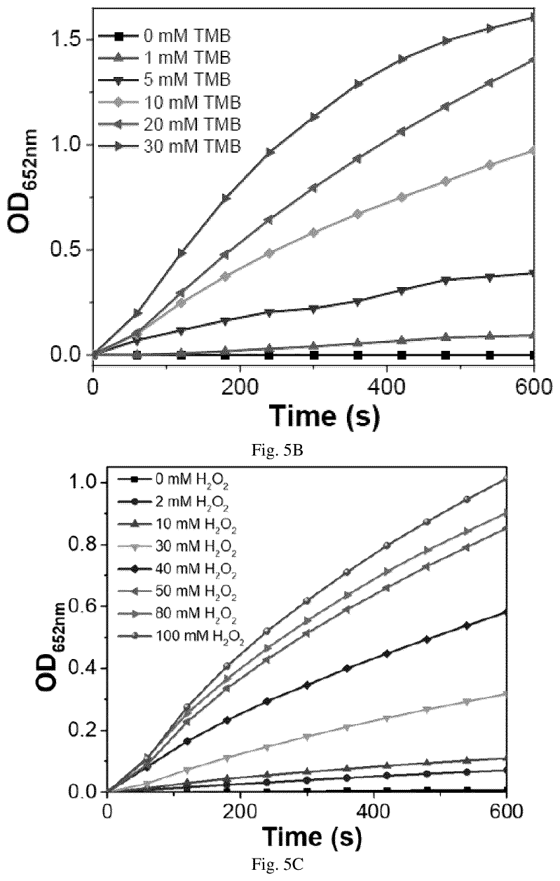

The carbon-based material acts as the host, providing a stable structure and surface for the guest, which is the single atom catalyst. With reference to FIG. 4C, the host-guest method allows for precise control over the dispersion and stability of the single atom catalyst, enhancing its catalytic activity. Without intending to be bound by theory, it is believed that the carbon-based host material provides a supportive environment for the single atom catalyst, preventing aggregation and improving its performance. By utilizing carbon and calcium sources in the host-guest method, a single atom nanozyme materials with enhanced catalytic properties can be created.

In an embodiment herein, the nitrogen-doped carbon material is the zeolitic imidazolate framework (ZIF), which can be selected from the group of from ZIF-8, ZIF-67, and a combination thereof, and the ZIF can be prepared by
(1) providing a first mixture comprising a Zn source;
(2) providing a second mixture comprising 2-methylimidazole; and
(3) mixing the first mixture and the second mixture by stirring to provide particles of the zeolitic imidazolate framework.

The first mixture and the second mixture are solutions including organic solvents such as methanol. For example, the Zn source (for example, 811 mg of zinc nitrate hexahydrate) can be dissolved in methanol (for example, 50 mL). 2-methylimidazole (for example, 1627 mg) can be dissolved in methanol (for example, 50 mL). The solution of the second mixture is poured into the solution of the first mixture quickly and stirred vigorously for, for example, 1 hour. Then, the resultant particles are separated by centrifugation, and washed with methanol several times to provide ZIF powder, which is then dried overnight at 60° C.

In some embodiments, the method further includes the step of introducing Ca²⁺ is absorbed into the solution of ZIF such as ZIF-8 to form Ca/ZIF-8 by means of ion exchange procedure.

In an embodiment herein, the pyrolysis process is performed by heating the precursor mixture to a temperature of about 900° C. to 1010° C., for example, about 905° C., about 910° C., at a rate ranging from greater than 0 to about 10° C./min in an inert atmosphere. For example, the heating rate could be about 1° C./min, 2° C./min, 3° C./min, 4° C./min, or 5° C./min.

In an embodiment herein, the method further comprises centrifuging and drying the precursor mixture in vacuum at 65° C. overnight prior to the pyrolysis process.

In an embodiment herein, the method further comprises an etching step using HCl to purify the single atom nanozymes after the pyrolysis process.

In an embodiment herein, the zeolitic imidazolate framework used for the method is ZIF-8 or ZIF-67, and the alkaline earth metal source comprises Ca.

In an embodiment herein, the organic ligands of ZIF-8 or ZIF-67 are transformed to an N-doped carbon structure, and the Ca atoms are trapped by the N-doped carbon structure during the pyrolysis process. Without intending to be bound by theory, it is believed that such N-doped carbon structure is benefit for easy anchoring of metals.

In an embodiment herein, the pyrolysis process is performed under Ar atmosphere. In some embodiments, the Ar gas is supplied at a rate of from about 5 mL/min to about 10 mL/min.

In some embodiments, alkaline earth metal single-atom nanozymes are prepared by host-guest method utilizing carbon and calcium sources. The alkaline earth metal includes calcium, magnesium, barium, etc. The carbon materials may be ZIF-8, ZIF-67, carbon fiber, carbon nanotube, graphene, carbon black, and reduced graphene oxide, etc. The carbon materials and the alkaline earth metal are mixed by stirring, for example, for more than 0.1 min. After stirring, the mixed solution was transferred to an airtight container, for example, a Teflon-lined stainless-steel autoclave. The container was heated to above 10° C. at a rate of 4° C./min, then cool to room temperature to obtain a gel-like product with a viscosity of about 2.4˜3.0 dl/g. The airtight container heated rate is greater than 0.1° C./min. The nanocrystals were then separated from the gel by centrifugation at above 100 rpm and then washed with methanol several times. The nanocrystals were heated at above 1° C. at a rate of >0.1° C./min and maintained at that temperature for more than 0.1 hours in a tube furnace filled with Ar/N₂. The resulting products were washed sequentially with pickling, DI water and finally dried in vacuum overnight at 60° C. at above 1° C. to obtained final products in powder form.

The calcium single atom nanozyme materials are obtained by a simple method of room temperature stirring and high temperature calcination. The enzyme catalytic system constructed using this material can be applied in antibacterial and antitumor applications. In terms of catalytic activity, the synthesized calcium single atom catalysts with a well-defined single structure can mimic the active sites of natural metalloproteases with the help of their atomically dispersed active sites and well-defined structures, thus improving their catalytic ability as nanozymes. Single-atom catalysts demonstrate great potential for understanding the catalytic mechanism of nanozymes and bridging the gap between natural enzymes and nanozymes. The introduction of calcium single-atom is more effective in enhancing the specific adsorption of hydrogen peroxide by the single-atom nanozymes catalysts, thus selectively improving their activity as peroxidase-like enzymes and bridging the gap between the specificity of single-atom nanozymes and natural enzymes, which can play a positive role in achieving efficient antibacterial and antitumor activities. The prepared materials show a record-high-rate constant efficiencies.

An embodiment herein relates to a method of tumor catalytic therapy comprising administrating a nanozyme comprising a single-atom alkaline earth metal loaded on a nitrogen-doped carbon material to a subject in need thereof, wherein the alkaline earth metal is selected from the group consisting of calcium, magnesium, barium, and a combination thereof, wherein the carbon material is selected from the group consisting of zeolitic imidazolate framework (ZIF), carbon fiber, carbon nanotube, graphene, carbon black, reduced graphene oxide, and a combination thereof.

It is known that nanozymes-based tumor catalytic therapy, in which nanozymes specifically trigger enzyme activity in tumor site to produce toxic reactive oxygen species (ROS) for killing tumor cells, represents a revolutionary anti-tumor approach. ROS are a class of oxygen-containing atoms or atomic groups that contain lone electrons and are chemically active, including superoxide anion, H₂O₂, singlet oxygen, hydroxyl radical (·OH), alkane peroxide radical and lipid peroxidation free radicals. ROS can kill tumor cells by mediating the damage of DNA, protein and other chemical substances. The high H₂O₂, hypoxia and acidic environment in solid tumors can make tumors resistant to radiotherapy, chemotherapy and photodynamic therapy and have low sensitivity. Therefore, regulating the balance between hypoxia and ROS may lead to tumor cell death. For nanozymes, oxidative stress can be induced to kill tumor cells by producing ROS through peroxidase (POD) and oxidase (OXD) activities. Since 2007, Fe₃O₄ nanoparticles have been reported to mimic the activity of natural peroxidase, additionally hundreds of nanomaterials have been found to mimic the enzyme activities of POD, OXD, catalase (CAT), glucose oxidase (GOx), glutathione peroxidase (GPx), superoxide dismutase (SOD) and uricase.

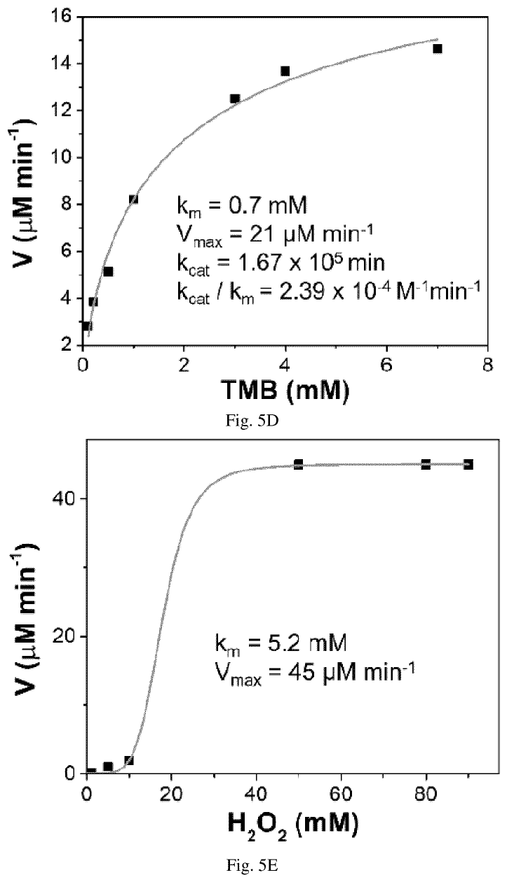

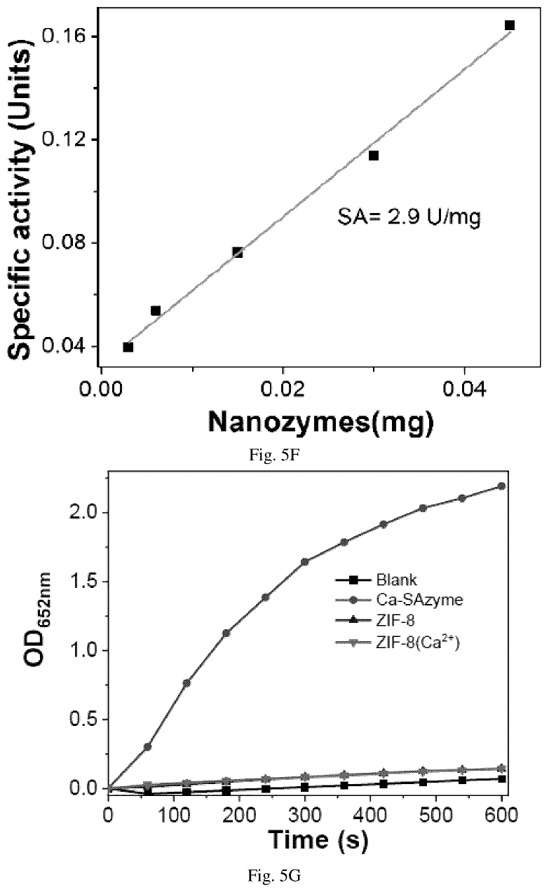

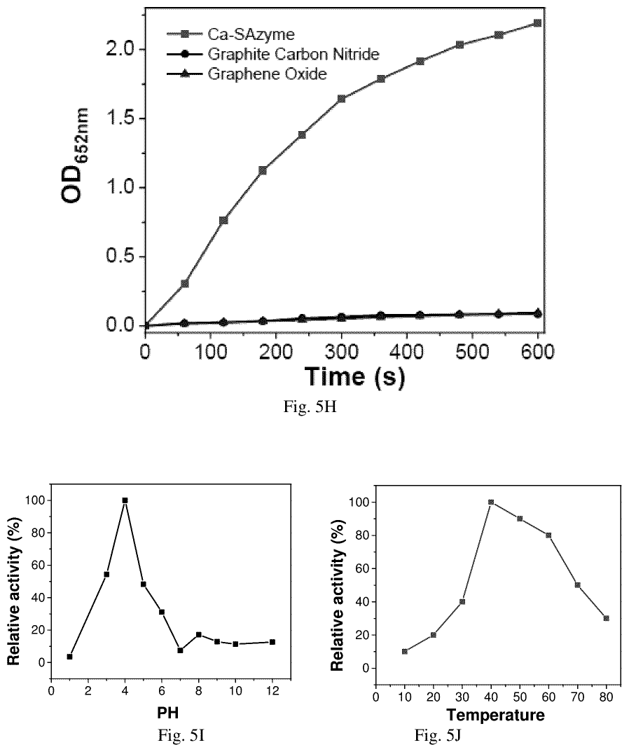

Without intending to be bound by theory, it is believed that the method of tumor catalytic therapy according to this invention can provide efficient catalytic therapy of gliomas and other refractory tumors, with minimal side effects. The present invention is mainly applied to antibacterial and antitumor applications, and a highly selective and sensitive colorimetric sensing platform was constructed using the specific and efficient peroxidase-like activity of calcium single atom nanozyme. With reference to FIG. 5F, the present invention makes full use of the unique structure and highly efficient enzymatic activity of calcium single atom nanozymes to overcome the problems of easy inactivation, poor stability and high cost of natural enzymes, and effectively improves antibacterial and antitumor capabilities.

Without intending to be bound by theory, it is believed that the engineered Ca-centered single-atom nanozyme (Ca-SAzymes) of this invention exhibits comparable peroxidase-like catalytic (POD) activity and kinetics to natural enzymes developing. It is also believed that, at GBM sites, Ca-SAzymes can generate a large amount of reactive oxygen species (ROS) through POD catalytic activity. This is also supported by our DFT calculations, which show a high selectivity for ·OH. Moreover, as an exotic Ca supplier, Ca-SAzymes cause mitochondrial Ca overload, which further amplifies the oxidative stress. More impressively, Ca-SAzymes can act as an immunoadjuvant to awaken innate immunity by stimulating the tumor to produce cytokine, which promotes polarization of tumor-associated macrophages to reverse the immunosuppressive microenvironment. Consequently, the designed Ca-SAzymes show promising potential for developing artificial enzymes that can overcome the adverse factors of GBM treatments and other refractory microenvironmental immunosuppressive tumors.

In an embodiment herein, the tumor for catalytic therapy is a refractory microenvironmental immunosuppressive tumor selected from the group of bladder cancer, bone tumor, brain cancer, breast cancer, and a combination thereof.

In an embodiment herein, the tumor is brain cancer. In particular, the brain cancer can be selected from the group of acoustic neuroma, astrocytoma, chordoma, CNS lymphoma, craniopharyngioma, brain stem glioma, ependymoma, mixed glioma, optic nerve glioma, subependymoma, medulloblastoma, meningioma, metastatic brain tumors, oligodendroglioma, pituitary tumors, Primitive Neuroectodermal (PNET), Other Brain-Related Conditions, schwannoma, and a combinaiton thereof. In some embodiments, brain cancer can be selected from the group of astrocytoma, ependymomas, glioblastoma, oligodendroglioma, medulloblastoma, and a combination thereof. More specifically, brain cancer is glioblastoma (GBM). Without intending to be bound by theory, it is believed that glioblastoma (GBM) is one of the most lethal neurological malignancies, with a median survival of only 15 months following surgical resection, postoperative radiotherapy, and adjuvant chemotherapy. Despite extensive efforts, emerging treatment strategies have shown limited success in improving outcomes. The resistance of GBM to treatment is attributed to several intrinsic features, including the heterogeneity of glioblastoma, the uniqueness of the glioma immune microenvironment, the infinite self-renewal capacity of glioma stem cell (GSC), and the resistance of the blood-brain barrier (BBB). Even the complex glioma microenvironment holds a glimmer of promise for tumor therapy. With the exploration of the tumor microenvironment, it is conceived that in glioma tissues, vigorous aerobic glycolysis will result in massive lactic acid accumulation and local intercellular mild acidosis (pH˜6.0). Meanwhile, relatively high concentrations of H₂O₂ (˜50-100 μM) have been found in tumor cells, generating from the dismutation of super anionic radicals by superoxide dismutase (SOD). These intrinsic tumor characteristics of the glioma microenvironments constitute the antecedent conditions for tumor-specific catalytic therapy and thus establish a profound basis for the development of new treatment methods for glioma.

In an embodiment herein, the nanozyme is administrated at a therapeutically effective amount of from about 2 mg to about 3 mg per kg body weight

In an embodiment herein, the loading percentage of the alkaline earth metal in the nanozyme ranges from greater than 0 to about 2.0 wt %. For example, the loading percentage can be about 0.5 wt %, about 0.8 wt %, about 0.9 wt %, about 1.0 wt %, about 1.5 wt %, about 1.8 wt %.

In an embodiment herein, the nanozyme has comparable peroxidase-like catalytic (POD) activity and kinetics to natural enzymes.

In an embodiment herein, the nanozyme acts as an immunoadjuvant to awaken innate immunity by stimulating the tumor to produce cytokine.

In an embodiment herein, the nanozyme induces tumor cell apoptosis via elevating ROS and reversing glioblastoma immune microenvironment.

In an embodiment herein, the nanozyme acts as an immunoadjuvant to awaken innate immunity by stimulating the tumor to produce cytokine, which promotes polarization of tumor-associated macrophages to reverse the immunosuppressive microenvironment.

In an embodiment herein, the nanozyme, as an exotic Ca supplier, causes mitochondrial Ca overload, which further amplifies oxidative stress in tumor.

### EXAMPLES

### 1. Materials

All reagents in the examples are used directly without further purification. Anhydrous calcium chloride (CaCl₂)), concentrated sulfuric acid (H₂SO₄≥98%) and potassium permanganate (KMnO₄) are purchased from Sinopharm Chemical Reagent (Shanghai, China). Graphite powder, sodium nitrate (NaNO₃), and 30% hydrogen peroxide (H₂O₂) are purchased from Macklin Reagent Co., Ltd. Ammonium sulfite monohydrate (NH₄)₂SO₃·H₂O) is purchased from Aladdin Reagent Co., Ltd. Nafion solution is purchased from Alfa Aesar. Deionized water used for all experiments is prepared using an ultrapure water generator.

### 2. Methods

### Characterization

Transmission electron microscopy (TEM) images, scanning transmission electron microscopy (STEM) images, and the selected area energy dispersive X-ray elemental mapping spectroscopy (EDS) were carried out using a JEM-2100F/HR transmission electron microscope at an accelerating voltage of 200 kV. High-angle annular dark field (HAADF) scanning transmission electron microscopy (STEM) images were detected using a JEOL JEM-ARM200F microscope incorporated with a spherical aberration correction system for STEM. X-ray diffraction (XRD) patterns were recorded on a BRUKER-D8 X-ray diffractometer using Cu Kα radiation (0.15418 nm). Raman spectra were performed using a Lab RAM high-resolution (HR) evolution Raman spectrometer operating at 514 nm. X-ray photoelectron spectroscopy (XPS) analyses were performed using an ESCALAB250 spectrometer equipped with a monochromatized Al Kα (1486.6 eV) source. The survey spectra were recorded in a 0.5 eV incremental with a pass energy of 140 eV. Detailed scans were recorded in a 0.1 eV incremental with a pass energy of 140 eV. The elemental spectra were all corrected using the C1s peak at 284.8 eV. The elemental analyses were conducted with an inductively coupled plasma optical emission spectrometer (ICP-OES) Optima 8000. The fluorescence spectrum was performed with a Fluorescence Detector (RF-10A, Shimadzu, Japan). The time-resolved photoluminescence (TRPL) decay spectra were recorded at 475 nm with 375 nm excitation on an Edinburgh FLS9800. Fourier transform infrared (FT-IR) spectra were recorded in the wavenumber range of 500-4000 cm-1 using a Nicolet iS10 (Thermo Fisher, USA) infrared spectrometer with a DTGS detector. X-ray absorption near edge spectroscopy (XANES) and extended X-ray absorption fine structure (EXAFS) spectroscopy were performed in transmission mode with the beamline XAFCA in a Singapore Synchrotron Light Source (SSLS). The electron storage ring of SSLS was operated at 700 MeV with a maximum current of 200 mA. An Si (111) double crystal was used to obtain a mono-chromate X-ray beam.

### DFT Methods

The first principles are employed to perform all Spin-polarization density functional theory (DFT) calculations within the generalized gradient approximation (GGA) using the Perdew-Burke-Ernzerhof (PBE) formulation. The projected augmented wave (PAW) potentials have been chosen to describe the ionic cores and take valence electrons into account using a plane-wave basis set with a kinetic energy cutoff of 450 eV. Partial occupancies of the Kohn-Sham orbitals were allowed using the Gaussian smearing method and a width of 0.05 eV. Electronic energy was considered self-consistent when the energy change was smaller than 10⁻⁵ eV. Geometry optimization was considered convergent when the energy change was smaller than 0.04 eV Å⁻¹. The vacuum spacing in a direction perpendicular to the plane of the structure is 15 Å. The Brillouin zone integration is performed using 3×3×1 Monkhorst-Pack k-point sampling for a structure. Finally, the adsorption energies (Eads) were calculated as Eads=Ead/sub−Ead−Esub, where Ead/sub, Ead, and Esub are the total energies of the optimized adsorbate/substrate system, the adsorbate in the structure, and the clean substrate, respectively. The free energy was calculated using the equation:

$$G = E + ZPE - TS where G , E , ZPE , and TS are the free energy , total energy from DFT calculations , zero - point energy , and entropic contributions , respectively .$$

### GSC Isolation and Cell Culture

Briefly, the specimens were cut into small pieces and digested into single cells with Accutase (Sigma), and red blood cells were lysed by Red Blood Cell Lysis Buffer (Solarbio). Cell suspensions were then passed through a 70 μm stainless steel mesh and re-cultured in the serum-free stem cell medium. The primary glioma spheres (GSC21 and GSC22) were all cultured in DMEM/F-12 medium (Gibco) containing 2% (vol/vol) B27 supplement (Gibco), epidermal growth factor (EGF, 20 ng/ml, Peprotech), basic fibroblast growth factor (bFGF, 20 ng/ml, Peprotech) and heparin (2.5 μg/ml, Sigma). Only early passage GSC cells were used for the study. Thp-1 was purchased from the National Collection of Authenticated Cell Cultures (NCACC, Shanghai, China) and were cultured in RPMI1640 (Gibco), containing 10% FBS and 1% penicillin/streptomycin. All cells mentioned above were cultured at 37° C. with 5% CO₂.

### Cytotoxicity

MTS kit and flow cytometer were employed to analyze cytotoxicity. Briefly, GSC21 and GSC22 cells (glioma stem cells from surgical resection tissues of two patients) were plated in 96-well plates with a density of 5*10³ cells per well and cultured in 100 μl culture medium for 24 h before the addition of Ca-SAzymes. Wells with culture medium-treated cells only were defined as 100% viability and blank wells with media were defined 0% viability. After incubation for various periods at 37° C., the supernatant was removed and 100 μl of medium and 10 μl MTS solution were added into each well. After incubation for another 2 hours in an incubator, the absorbance at 490 nm was determined to calculate the survival rates in different environments. For the killing mechanism study of Ca-SAzymes to glioma stem cells, GSC21 cells were co-incubated with Fer-1 and MA and cell viability was measured by MTS assay, equally. For flow cytometer analysis, procedures were similar to the above except that cells were cultured in 6-well plates and incubated with Ca-SAzymes for 48 h. At the end of co-incubation, cells were treated with Annexin V/PI for flow cytometer,

### Intracellular ROS Measurement

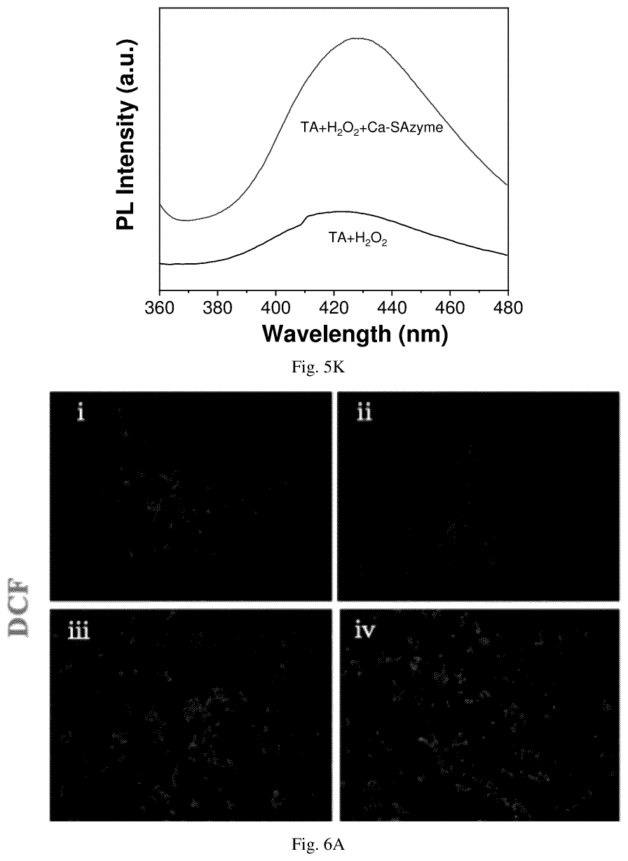

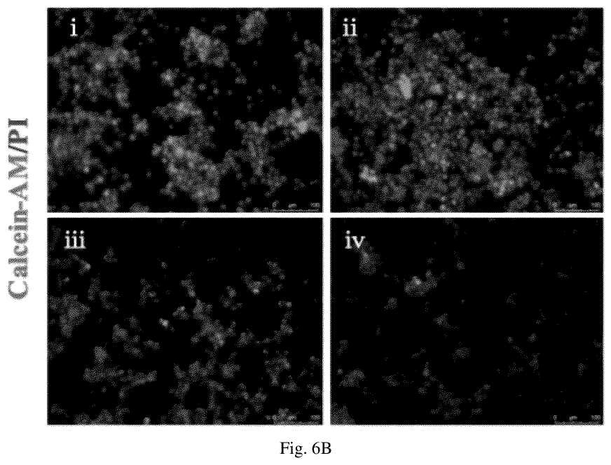

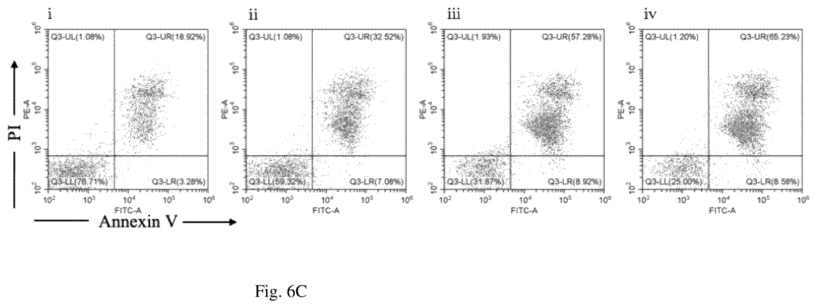

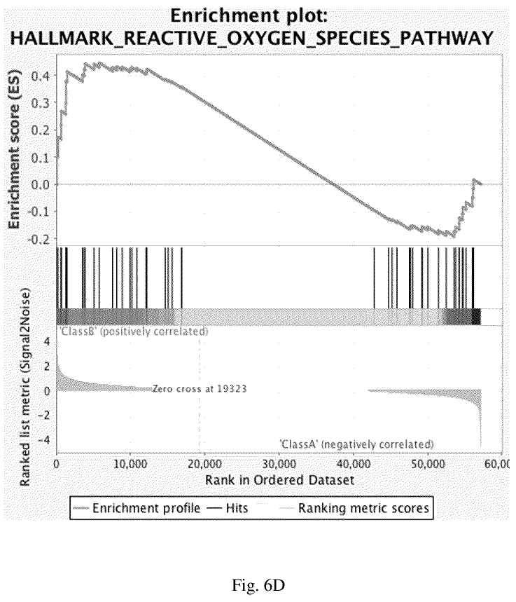

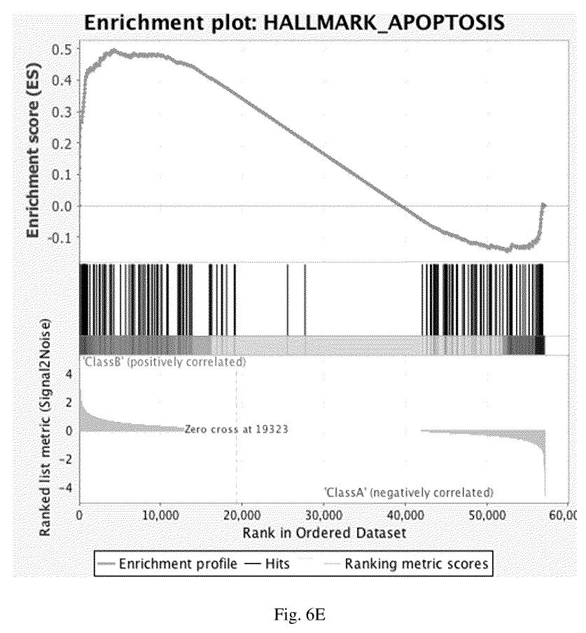

A fluorescent probe, DCFH-DA was used to measure the intracellular generation of ROS by Ca-SAzymes. GSC21 cells were planted in 6-well plates (1*10⁶ cells per dish) with laminin to adherent growth. Then GSC21 cells were incubated with different concentrations of Ca-SAzymes in the absence or presence of 100 μM H₂O₂ for 24 h. Subsequently, the culture medium was replaced by fresh DCFH-DA containing medium (10 μM) for 20 min, followed by washing with PBS buffer. Finally, the ROS fluorescence signals were observed by fluprescence microscope. The condition for DCFH-DA cell-staining: Ca-SAzymes: 0, 20, 50, 200 μg mL⁻¹ (corresponding in turn to diagram i, ii, iii, iv in FIG. 6A), H₂O₂: 100 μM, pH=7.4.

### Western Blotting

GBM cells were lysed via vigorous sonication on ice bath and protein concentration was determined by Bradford assay. Thereafter, immunoblotting of cell lysates was performed using antibodies to Calpain-1 (ab108400, Abcam), Cleaved-caspase 3 (9664, CST), MRC1 (18704-1-AP, Proteintech), NOS₂ (AF7281, Beyotime), and GAPDH (60004-1, PTG) according to standard protocols. Secondary antibody (1:5000,) for another 1 h at room temperature, and visualized using Western Blotting Luminol Reagent (Santa Cruz Biotechnology, CA, USA).

### RNA Extraction and RT-qPCR

Total RNA was extracted from GSC21 cells and macrophages using Trizol reagent following the manufacturer's guideline (Invitrogen, USA). mRNA was converted to cDNA using PrimeScrip RT Master Mix (RR036A, Takara, Shiga, Japan), and then expression levels of analysed genes determined using the SYBR Premix Ex Taq Kit (RR420A, Takara) under amplification conditions. GAPDH was used as the reference gene for normalization, and mRNA abundance was quantified using the threshold cycle method. Each reaction was performed in triplicate. Primers are listed in Table 1 below.

TABLE 1
PCR Primers and siRNA sequences
qPCR Primers

| Primer | Forward (5′-3′) | Reverse (5′-3′) |
|---|---|---|
| IFN-γ | TCGGTAACTGACTTGAATGTCCA | TCGCTTCCCTGTTTTAGCTGC (SEQ ID |
| | (SEQ ID No. 1) | No. 2) |
| NOS2 | TCCCACCTGACCTTGTGCTT (SEQ | CAGGGCGTACCACTTTAGCTC (SEQ |
| | ID No. 3) | ID No. 4) |
| Il6 | AACAAATTCGGTACATCCTCGAC | ATTTTCACCAGGCAAGTCTCC (SEQ |
| | (SEQ ID No. 5) | ID No. 6) |
| TNF-α | TCGAACCCCGAGTGACAAGCC | GTTCTTATACTCGGGCCACAC (SEQ |
| | (SEQ ID No. 7) | ID No. 8) |
| GAPDH | ACGACCACTTTGTCAAGCTC (SEQ | CTGTTGCTGTAGCCAAATTCGTT (SEQ |
| | ID No. 9) | ID No. 10) |

Single Sample Gene Set Enrichment Analysis (ssGSEA) of ROS Metabolism

The Cancer Genome Atlas (TCGA) transcriptome pan-cancer data (2016 Dec. 29 version) and clinical pan-cancer data (2018 Sep. 13 version) were downloaded from Xena Website (https://xenabrowser.net/). 34 reactive oxygen species metabolism related gene ontology biological process signature (c5. go. bp. v7.5.1.s1. symbolsrsion) were downloaded from the MSigDB website (https://www.gsea-msigdb.org/gsea/msigdb/index.jsp). The ssGSEA method was chosen to evaluate each signature activity in different TCGA samples using the GSVA R package. Box plot was used to show ROS metabolism in different individuals among different cancer types by illustrating the ssGSEA value of GOBP_REACTIVE_OXYGEN_SPECIES_METABOLIC_PROCESS.t-distributed stochastic neighbor embedding (tSNE) was used to reduce the dimension of 34 ROS-related signature values among TCGA samples by using the Rtsne R package.

### Go Analysis

DESeq2 R package was used to calculate differential gene expression (DGE) between the NC group and the Ca-SAzymes group. clusterProfiler R package was used to perform gene ontology (GO) analysis of the DGE between the NC group and the Ca-SAzymes group. GOplot R package was used for illustrating GO analysis result.

### TEM Analysis of Mitochondria

GSC21 cells were first incubated with 50 μg/ml Ca-SAzymes for 24 h and subsequently fixed in 4% glutaraldehyde after trypsinization, rinsed in PBS and finally centrifuged at 1,200 rpm for 5 min. The collected cells were fixed in 2% osmium tetroxide in 0.1 M PBS at 4° C. for 1 h, subsequently dehydrated in ethanol followed by acetone and embedded in LX-112. Ultrathin sections were cut with a Daitome Diamond slicer and examined under a hitachi transmission electron microscope.

### In Vivo Anticancer Evaluation

Female immunocompetent BALB/c mice and C57BL6 (4-6 weeks, 18-20 g) purchased from Beijing Vital River Laboratory Animal Technology were used to establish intracranial GBM xenografts. A total of 2*10⁵ GSC21 cells with luciferase was intracranially injected into mice to create a GBM-bearing murine model. For intratumoural injection studies, 15 mice bearing GSC21 cells were randomly assigned to 3 groups (n=5 mice per group) 3 days after tumour implantation. Mice in the first group were in situ injected with PBS and ZIF-8, second group mice were injected with Ca-SAzymes dissolved in PBS at a dose of 2 mg per kg body weight at 3 days after tumour implantation. Mice in the third were in situ injected with the same Ca-SAzymes from day 9. For the groups treated with Ca-SAzymes materials, 3 injections were administered in total, at 2-day intervals.

The mice's body weight was measured every 5 days to observe the health status. On day 21, we performed live imaging of small animals by injecting substrates. For biosafety analysis, the major organs (heart, liver, spleen, lung and kidney) were harvested for H&E staining after mice sacrificed, and the brains were collected for H&E staining investigation to direct compare the tumour size.

### Example 1

### Synthesis of Ca-SAzymes

This example illustrated the fabrication of Ca-based nanozymes by atomically confined Ca in nitrogen-doped graphene (Ca-SAzyme).

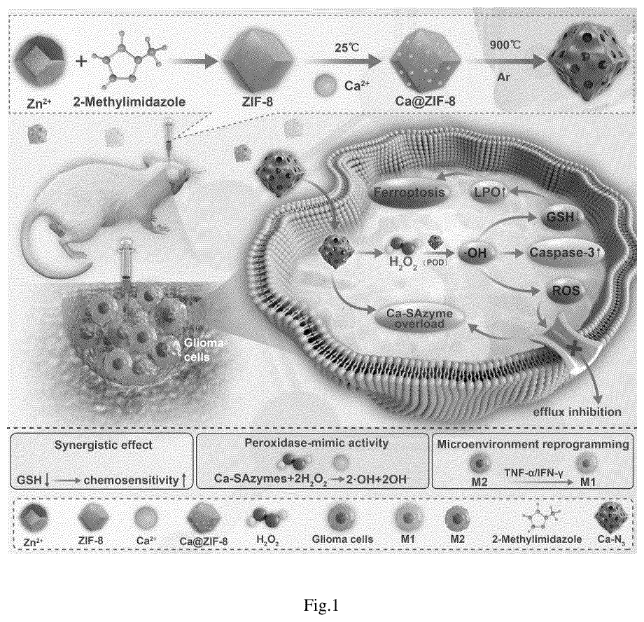

Specifically, the upper part of FIG. 1 diagrammatically depicts the synthesis of Ca-SAzyme.

First, zeolitic imidazolate framework-8 (ZIF-8) was made by combining nitrate hexahydrate with 2-methylimidazole in the presence of methanol at ambient temperature. In a typical synthesis, 811 mg of zinc nitrate hexahydrate was dispersed into 50 mL methanol solution, donated solution A. Solution B consists of 1627 mg of 2-methylimidazole dissolved in 50 mL methanol. After solution A and solution B are completely dissolved, B was poured into the Zn-based solution A quickly and vigorously stirred for 1 hour. The resultant particles were separated from the gel by centrifugation at 4000 rpm and washed with methanol 4 times. This procedure was repeated twice. The resultant ZIF-8 powder was dried overnight at 60° C.

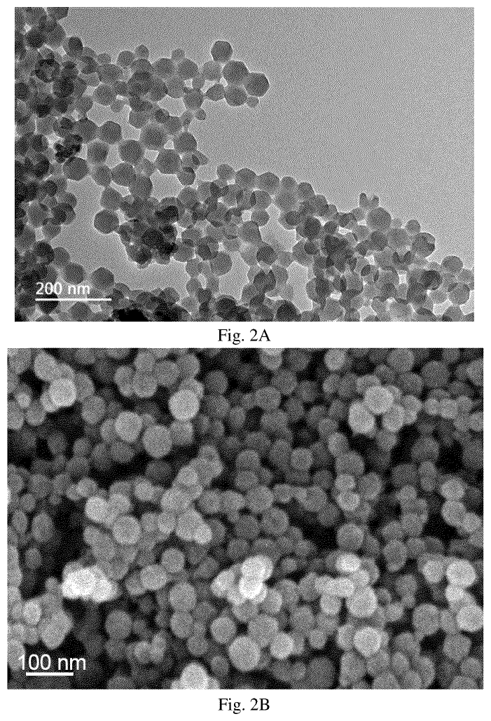

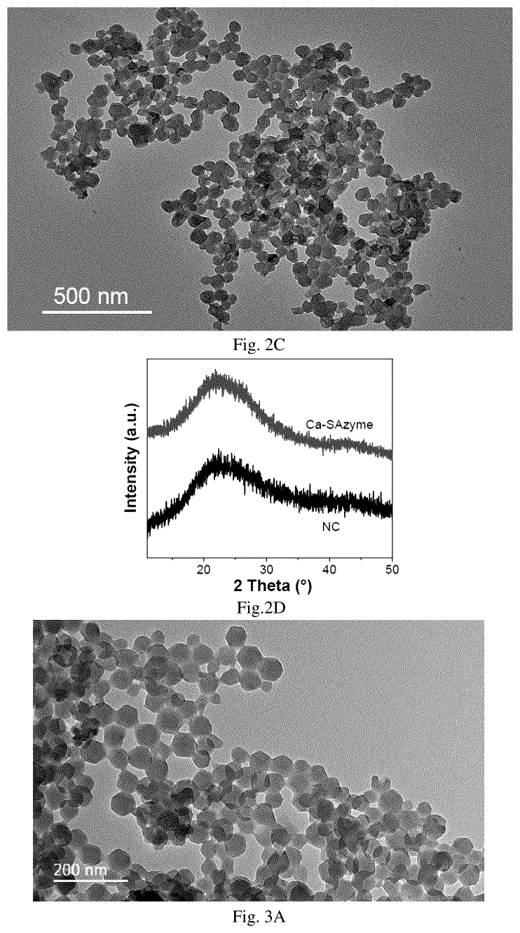

Then, using the ion exchange procedure, Ca²⁺ was absorbed into the ZIF-8 solution to form the Ca/ZIF-8 (FIG. 2C). In particular, the powder of ZIF-8 (0.20 g) was dispersed in water (10 mL) under ultrasound for 5 minutes at room temperature. After forming homogeneous mixture, CaCl₂) aqueous solution (1 g mL⁻¹, 500 μL) was injected into the mixed solution slowly under stirring. Next, the mixture was vigorously stirred for 1 h at room temperature so that the salt solution was absorbed completely. After centrifuging and drying in vacuum at 65° C. overnight, the sample was placed in a tube furnace and pyrolyzed to 900° C. (heating rate 5° C./min) for 2 h under Ar (10 mL/min) atmosphere to yield Ca-SAzyme. The organic MOF ligands were transformed to an N-doped carbon structure during the pyrolysis process, and the N-rich porous carbon trapped the Ca atoms to create the Ca-SAzyme.

The high-resolution transmission electron microscope (HR-TEM) and Scanning Electron Microscope (SEM) images of ZIF-8 show the uniform size distributions and pentagonal morphologies (FIGS. 2A and 2B).

The X-ray diffraction (XRD) pattern of Ca/ZIF-8 is consistent with ZIF-8, indicating that the addition of Ca does not affect the crystallization of ZIF-8. The XRD pattern of Ca-SAzyme (FIG. 2D) was similar to nitrogen-doped carbon (NC), with no Ca distinctive peaks, indicative of the poor crystallinity of Ca-SAzyme.

### Example 2

### Characterizations of Ca-SAzymes

Different methods were used to characterize the Ca-SAzymes as prepared in Example 1.

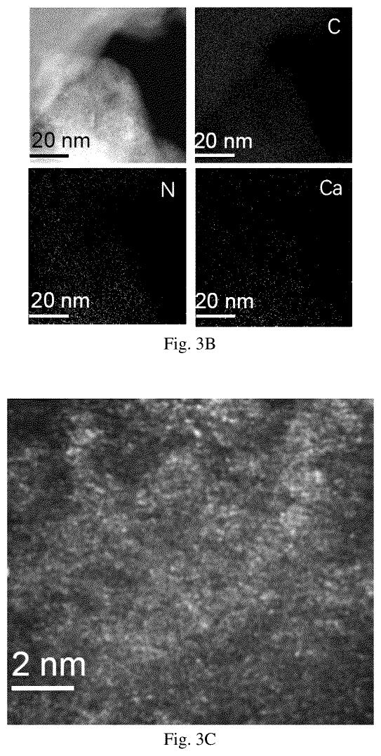

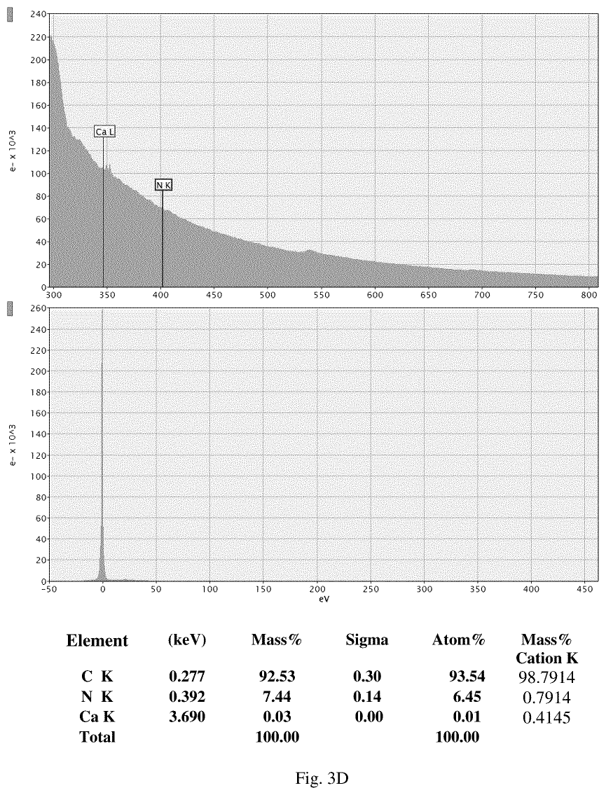

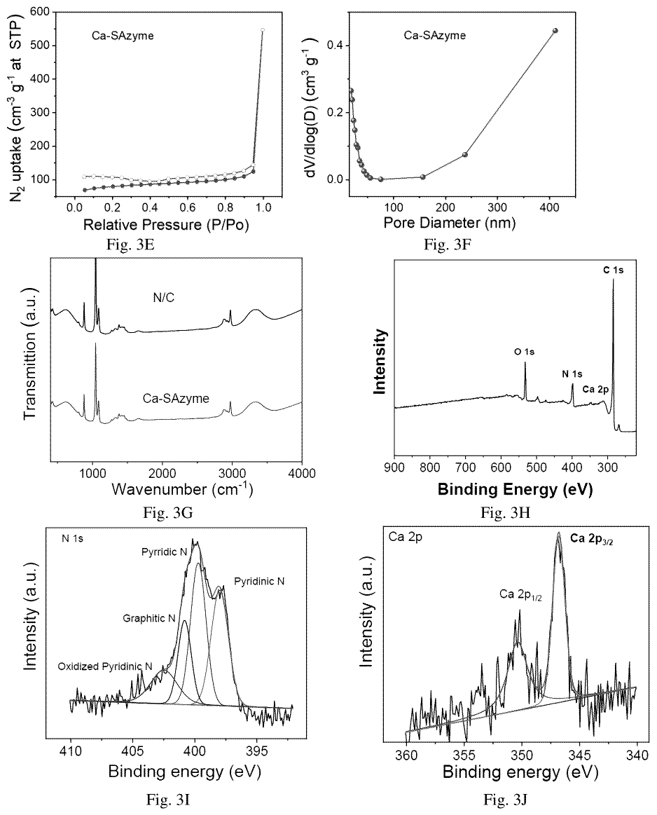

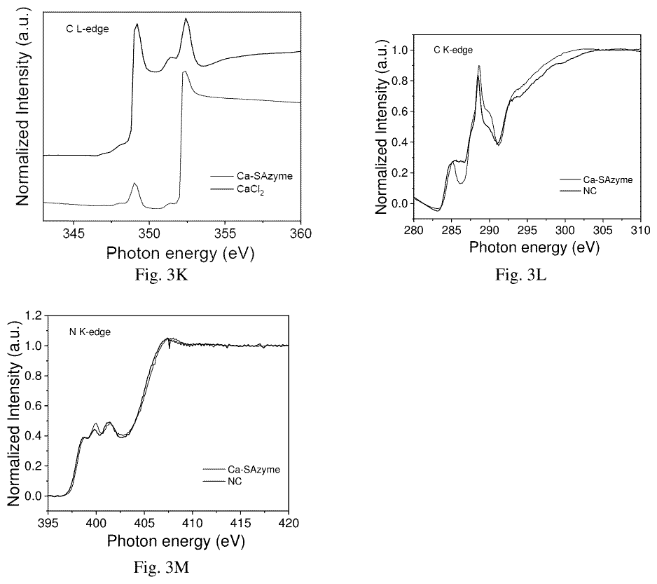

The HR-TEM image of the produced Ca-SAzyme revealed a polyhedral shape that is comparable to that of pure ZIF-8, indicating that no Ca-related nanoparticles were found in the materials (FIG. 3A).

From the aberration-corrected high-angle annular dark-field scanning transmission electron microscopy (HAADF-STEM) images, bright spots (highlighted by red circles) belonging to isolated Ca atoms distributed over NC can be recognized (FIG. 3C). The accompanying HAADF-STEM energy-dispersive X-ray spectroscopy (EDS) mapping pictures (FIG. 3B) demonstrated that Ca, C, and N were distributed uniformly throughout the substrate. Additionally, the existence of Ca and N components was verified using electron energy loss spectroscopy (EELS). The production of Ca-Nx Bonds is indicated by two peaks about 345 and 401 eV, respectively, known as the typical Ca L_{2,3} and N_{κ} edges (FIG. 3D). Brunauer-Emmett-Teller (BET) analysis and the corresponding pore size distribution (FIGS. 3E and 3F) revealed that Ca-SAzyme exhibited a porous structure with a large surface area, which was advantageous for exposing Ca active sites. FIG. 3G showed that Ca-SAzyme has similar transmission spectra of NC. According to

The coordinated environment and electronic information of Ca single atoms are analyzed for X-ray photoelectron (XPS) photoelectron and X-ray (XAS) absorption spectroscopy. The spectrum of the survey with the main peaks of C and some smaller peaks of N, O, and Ca confirms the presence of C, N, O, and Ca in Ca-SAzyme (FIG. 3H). The high-resolution N Is spectrum reveals that the Ca-SAzyme catalyst predominantly comprises pyridine nitrogen, with trace amounts of pyrrolic, graphite, and oxidized nitrogen species (FIG. 3I). Binding energy peaks 346.8 eV and 350.3 eV (FIG. 3J) may be attributed to Ca 2p3/2 and Ca 2p1/2 respectively under the NIST Photoelectric Spectroscopy database. The Ca 2p3/2 (344.9 eV) lack of metal spectrum shows that Ca-cations (shown in HAADF-STEM pictures) are Ca²⁺ in Ca-SAzyme. The L-edge X-ray absorption near-edge (XANES) structure (FIG. 3K) may be divided into the major peaks of the L₃ and L₂ edges, which well coincide with the HAADF and EELS analyses respectively. The substances C and N are primarily defined by the fine structure of fine X-ray absorption based on Synchrotron (NEXAFS). In the C-edge spectrums, 3 typical resonances are found in Ca-SAzyme and NC catalysts, approximately 285.1 eV (π*_{C═C}), 288.5 eV (π*_{C—N—C}), and 293.0 eV (σ*_{C—C}) (FIG. 3L). Three characteristic peaks corresponding to the π*-transition of pyridinic N, pyrrolic N, and graphitic N species, respectively, are located at roughly 398.2, 399.1, and 400.5 eV in the N K-edge spectra (FIG. 3M). Previous research has shown that pyridinic N or pyrrolic N species can donate one π-electron to the π conjugated structure, which is commonly thought of as a coordination site for atomically scattered Ca metals (Sun, J. et al. Atomically confined calcium in nitrogen-doped graphene as an efficient heterogeneous catalyst for hydrogen evolution. iScience. 24, 102728 (2021)). Furthermore, graphitic N may affect the electrical and geometric nanostructures of carbon substrates.

The Ca K-edge of XANES curves of Ca-SAzyme and CaO are shown in FIG. 4A.

TABLE 2
EXAFS fitting parameters at the Ca K-edge for various samples (S₀² = 0.780).

| | | | | | | |
|---|---|---|---|---|---|---|
| Sample [symbol] | Shell [symbol] | CN^{a} [symbol] | R(Å)^{b} [symbol] | σ²(Å²)^{c} [symbol] | ΔE₀(eV)^{d} [symbol] | R factor [symbol] |

Ca K-edge

[symbol]

| | | | | | | |
|---|---|---|---|---|---|---|
| CaO [symbol] | Ca—O [symbol] | 6.0 [symbol] | 2.332 ± 0.015 [symbol] | 0.0070 ± 0.0010 [symbol] | 9.51 ± 1.08 [symbol] | 0.006 [symbol] |
| Ca—CN [symbol] | Ca—O [symbol] | 1.32 ± 0.26 [symbol] | 2.281 ± 0.004 [symbol] | 0.0036 ± 0.0007 [symbol] | 2.65 ± 0.56 [symbol] | 0.008 [symbol] |
| | Ca—N [symbol] | 1.89 ± 0.33 [symbol] | 2.474 ± 0.002 [symbol] | 0.0036 ± 0.0007 [symbol] | | |

^{a}CN, coordination number;
^{b}R, distance between absorber and backscatter atoms;
^{c}σ², Debye-Waller factor to account for both thermal and structural disorders;
^{d}ΔE₀, inner potential correction;
R factor indicates the goodness of the fit.
S0² was fixed to 0.780.
Fitting range: 3.0 ≤ k (/Å) ≤ 12.0 and 1.0 ≤ R (Å) ≤ 2.4 (CaO); 3.0 ≤ k (/Å) ≤ 1|0.4 and 1.0 ≤ R (Å) ≤ 3.0 (Sample Ca).

[symbol]

The Ca K-edge XANES spectra of CaO were utilized as a calibration reference material since calcium metal is extremely active in the air. FIG. 4A depicts the Ca-SAzyme XANES adsorption edge location being equal to that of CaO, which suggests that Ca metal atoms in Ca-SAzyme are in cationic states. The results of the XPS spectra agree with this. This visualization is shown in FIGS. 4B and 4C, respectively, for the Ca-SAzymes, which may be referred to as Ca EXAFS R-space and Ca K-edge EXAFS R-space. Ca-SAzyme has a distinct EXAFS curve than CaO. Ca-SAzyme R space plots reveal a strong peak at around 1.8 Å. CaO, on the other hand, exhibits two significant bonding characteristics at about 1.8 Å and 3.1 Å, which are attributable to the Ca—O and Ca—O—Ca bonds, respectively. Ca-main SAzyme's peak at around 1.8 Å is attributed to the creation of the Ca—O bond, which illustrated that the exit of Ca cations rather than compound. Atomic structural simulations reveal that the attached Ca single atoms are situated at the defects in NC generated from pyridine-N. (FIG. 4D and FIG. 2B). The fitting findings revealed that the Ca—N contribution to Ca-SAzyme had a coordination number (CN) of 3. This finding is consistent with DFT simulations indicating that single Ca atoms embedded in carbon's pyridinic N defects are stable.

### Example 3

### Performances for the Peroxidase (POD) Activity of Ca-SAzyme

The performances for the POD activity of Ca-SAzyme were tested using the product as prepared in Example 1.

### Methods

Using 3,5,3′,5′-tetramethylbenzidine (TMB) as a probe, the peroxidase (POD)-like activity of Ca/NG-SAzyme was evaluated at room temperature. In 0.01 M buffer, the test was carried out in the presence of H₂O₂. The Hitachi UV2010 spectrophotometer was used to monitor all kinetic data in a time-scan mode at 652 nm. To understand how concentration dependence the POD-like activity of Ca-SAzyme, a test was performed in which Ca/NG-SAzyme (in different concentrations, from 0 to 200 μg/mL) was mixed with various concentrations of H₂O₂ (1.0 mM), which was then followed by the addition of 1 mM TMB, the final 650 nm absorbance of the combination was detected.

For the determination of kinetic parameters, tests were conducted in a 0.01 M HAc—NaAc buffer containing 50 μg/mL PMCS, 20 mM H₂O₂, and a series of TMB concentrations ranging from 0 to 7 mM, or in 2 mM TMB, and a series of H₂O₂ concentrations ranging from 0 to 100 mM.

Production absorbance was measured across a range of reaction times. By the Michaelis-Menten equation, which explains the correlation between the rates of substrate conversion by Ca-SAzyme, the absorbance data was fitted to a catalytic model.

To determine the influence of pH and temperature on peroxidase activity of Ca-SAzyme, 100 μg Ca-SAzyme, 1 mM H₂O₂, and 1 mM TMB were added to each of the pH range of 1.0˜12.0 and the temperature range of the 10˜80° C. solutions of the HAc—NaAc buffer with or without PBS buffer. To measure the 650 nm absorbance of the final combination, the Hitachi UV2010 spectrophotometer was used to monitor. For the sake of comparison, the carbon framework (C—N) was doped with nitrogen.

The production of hydroxyl radicals was detected using the terephthalic acid photoluminescence test. 1 mM H₂O₂, 0.5 mM terephthalic acid were incubated for 5 minutes at 25° C. in 0.01 M HAc—NaAc buffer (pH 4.0). At 410 nm, the mixture was analyzed using a fluorescence spectrophotometer.

### Results

The peroxidase (POD) activity of Ca-SAzyme was evaluated in the catalytic oxidation of 3,3′,5,5′-tetramethylbenzidine (TMB) in the presence of H₂O₂ converted TMB to blue oxide, and the formation of TMB oxide increased with the concentrations of Ca-SAzyme, TMB, and H₂O₂ (FIG. 5A to 5C). Based on the velocity of the Michaelis-Menten constant (K_{M}) of Ca-SAzyme was calculated using the TMB oxidation velocity, yielding K_{M}=0.7 mM and Vmax=21 μM min⁻¹ for TMB, and K_{M}=1.78 mM and Vₘₐₓ=45 μM min⁻¹ for H₂O₂ (FIGS. 5D and 5E). Based on a TMB oxidation rate at varied Ca-SAzyme concentrations, FIG. 5F shows that the Ca-SAzyme specific POD-like value of the activity (SA) was determined to be 2.9 U/mg. One nanozyme activity unit (U) is defined as the amount of nanozyme that catalyses 1 μmol of product per minute. Furthermore, in the qualitative analysis experiments (FIG. 5G and FIG. 5H), which tested the same reaction conditions on both the Ca-SAzyme and other carbon materials (graphitic carbon nitride and graphene oxide), the Ca-SAzyme exhibited notably higher peroxidase-like activity than these other carbon materials. More importantly, when the Ca²⁺ into the ZIF-8 solution, there was no effect in peroxidase-like activity compared to ZIF-8, supporting the notion that the Ca-SAzyme could be used as a single-atom peroxidase mimic and no Ca²⁺ nanoparticles in the reaction system. Ca-SAzyme demonstrated good catalytic activity throughout a broad temperature range of 10-80° C. and pH range of 1-12, as shown in FIGS. 5I and 5J, which has an optimal temperature of approximately 40° C. and an optimal pH value of around 4. In addition, the production of ·OH about Ca-SAzyme was assessed by using terephthalic acid (TA) as a ·OH probe (FIG. 5K). As shown in FIG. 5F, the signal intensity increased with the presence of Ca-SAzyme, affirming that Ca-SAzyme could effectively produce ·OH.

### Example 4

### In Vitro Cytotoxicity and Cellular Distribution of Ca-SAzymes

The vitro cytotoxicity and cellular distribution of Ca-SAzymes prepared according to Example 1 were tested in this example.

Taking advantage of the peroxidase-like activity of Ca-SAzymes, which could catalytically generate abundant oxidative species selectively in a glioblastoma-acidic and H₂O₂ environment (FIG. 6A), the Ca-SAzymes is employed as a therapeutic strategy for glioblastoma cell suppression (FIG. 6B) and assessed its effectiveness. FIG. 6A shows the images of cells treated with Ca-SAzyme, H₂O₂ and their combination, in which the condition for DCFH-DA cell-staining

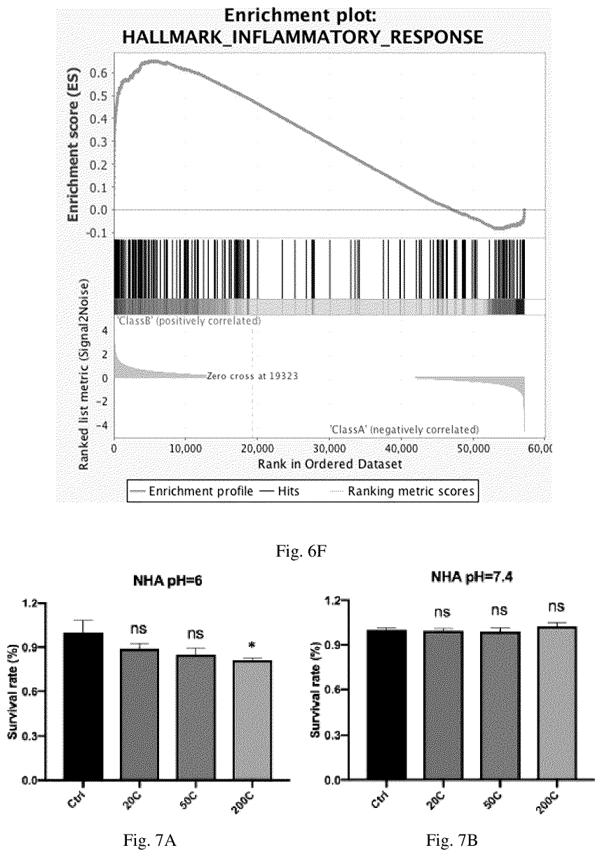

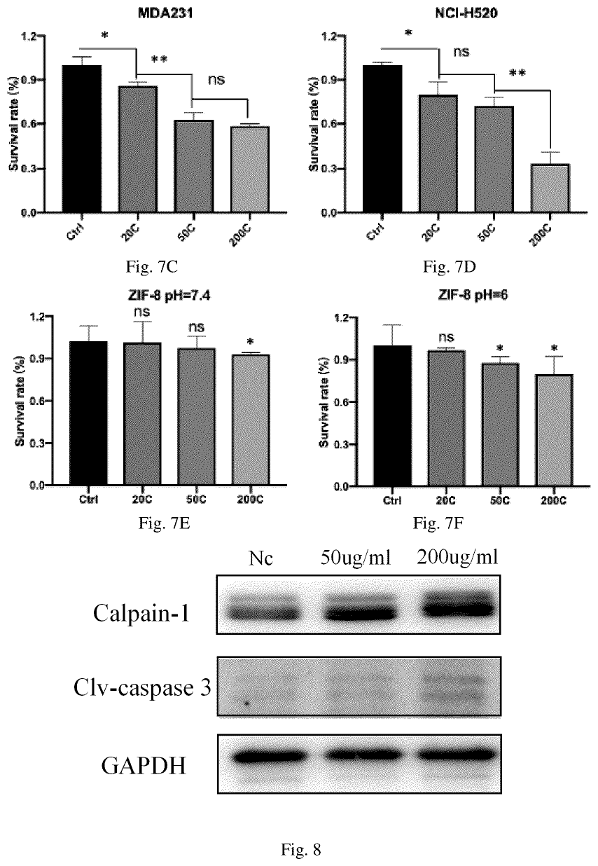

In glioblastoma cell viability assays, ZIF-8 without calcium atoms showed limited cytotoxicity. However, only ˜25% of glioblastoma cells from the #21 patient survived when treated with Ca-SAzymes (200 μg ml⁻¹) for 24 h (FIG. 6C). To further explore the multilevel damage caused by Ca-SAzymes, RNA sequencing of glioblastoma cells after treatments with Ca-SAzymes was performed. Based on RNA discrepancy, ROS, and apoptosis-related genes were also accumulated at the mRNA level by GSEA (FIGS. 6D and 6E), which matches the pattern at the cellular level. In addition to the verified cellular level damage, we also found extensive mobilization of the entire glioblastoma immune system through GSEA (FIG. 6F), including cytokines and pathways that activate the immune system and potentially alter the fate of tumors (FIG. 7A-7F). In FIGS. 7A-7F, data were presented as means±standard deviations; Student's t test; *means P<0.05, **means P<0.01.

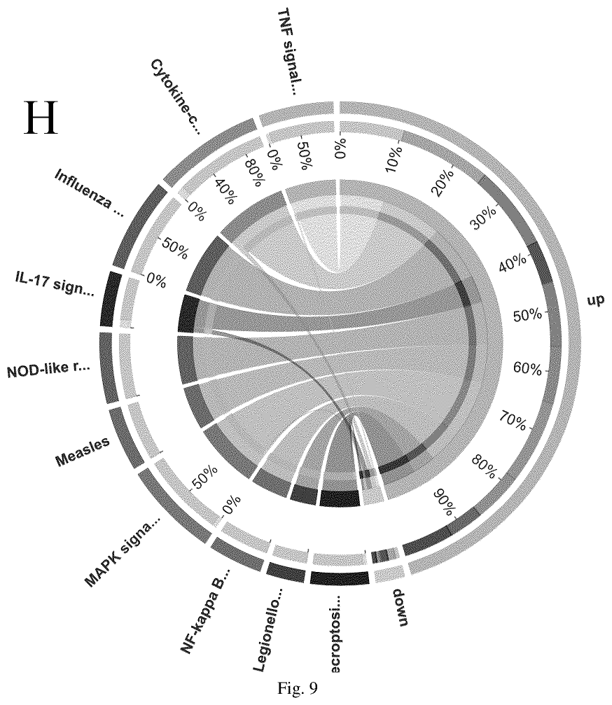

As discussed in the previous section, the POD-mimicking activity of Ca-SAzymes predisposes glioblastoma cells to severe apoptosis. Nonetheless, POD enzyme activity is by no means the only cause of glioblastoma cell death. The original design was based on the idea that calcium ions could affect mitochondrial homeostasis and lead to programmed glioblastoma, to enhance the tumor cytotoxicity through the synergistic effect of the metal ions and Fenton reaction. Calpain-1, a protein associated with the enrichment of calcium in cells, exhibited an elevating level after treatment with Ca-SAzymes and demonstrated the enrichment of intracellular calcium (FIG. 8). After a series of cascading reactions, it ultimately led to high expression of clv-caspase3 in glioblastoma cells, leading to an apoptotic pathway and cellular disruption. For further mechanistic analysis, the top 10 pathways based on the Kyoto Encyclopedia of Genes and Genomes (KEGG) analysis were constructed (FIG. 9). “TNF signaling pathway”, “Necroptosis” and some inflammatory pathways related genes were enriched by Ca-SAzymes, indicating tumor homeostasis might be disrupted by these pathways.

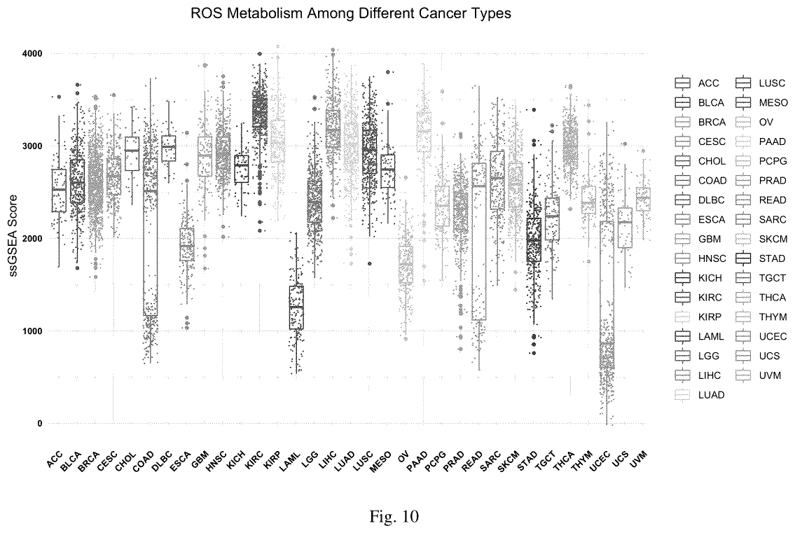

To investigate the metabolic capacity of glioblastoma, ROS metabolism genes from different cancer patients were integrated, which showed that glioblastoma had a high level of ROS metabolism (FIG. 10). The metabolic capacity of ROS was also unequal among patients in the group, which was reflected in the drug sensitivity result that disparate patients have disparate Ca-SAzymes sensitivity. The outcome of in vitro therapy in patient #21 was significantly better than in patient #22, indicating that patient #21 might be more suitable for Ca-SAzymes catalytic therapy (FIGS. 11A and 11B). In FIGS. 11A and 11B, values are presented as means±s.d. (experiments were performed in quadruplicates). *P<0.05, **P<0.01, ***P<0.001. Treatment of human glioblastoma cells and other tumor cells with Ca-SAzymes demonstrated a concentration-dependent and time-dependent tumor cell death (FIGS. 7A-7F and FIG. 12). To verify the mechanism of glioblastoma cell damage, antioxidants, and protein inhibitors were introduced to regulate cellular activities. It has been found that the oxidative damage caused by Ca-SAzymes could be adequately rescued via pre-incubation with α-Tocopherol. Since α-Tocopherol is fat-soluble, it is speculated that apart from the Fenton reaction-generated ·OH, lipid peroxidation (LPO) is a concomitant by process (FIG. 11C). As expected, ferroptosis-1 (ferroptosis inhibitor) could also prevent cell death caused by Ca-SAzymes, verifying the ferroptosis hypothesis.

### Example 5

### Glioblastoma Progression Inhibition of Ca-SAzymes by Activating Immune Microenvironment

The glioblastoma progression inhibition of Ca-SAzymes prepared according to Example 1 was tested in this example.

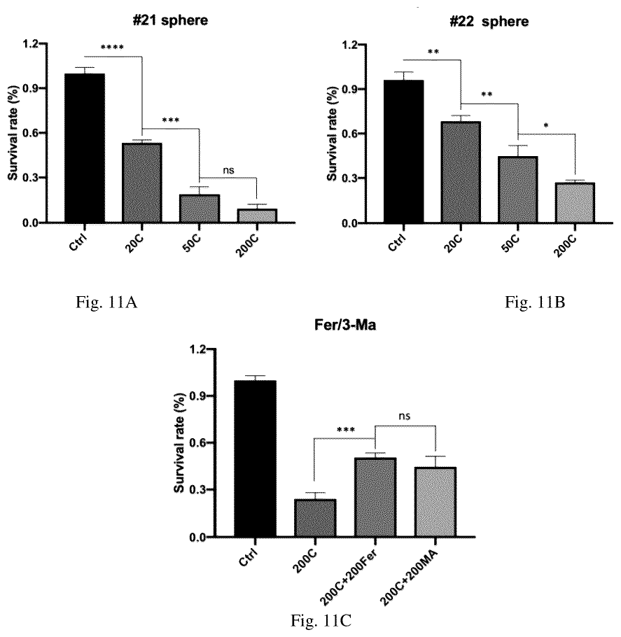

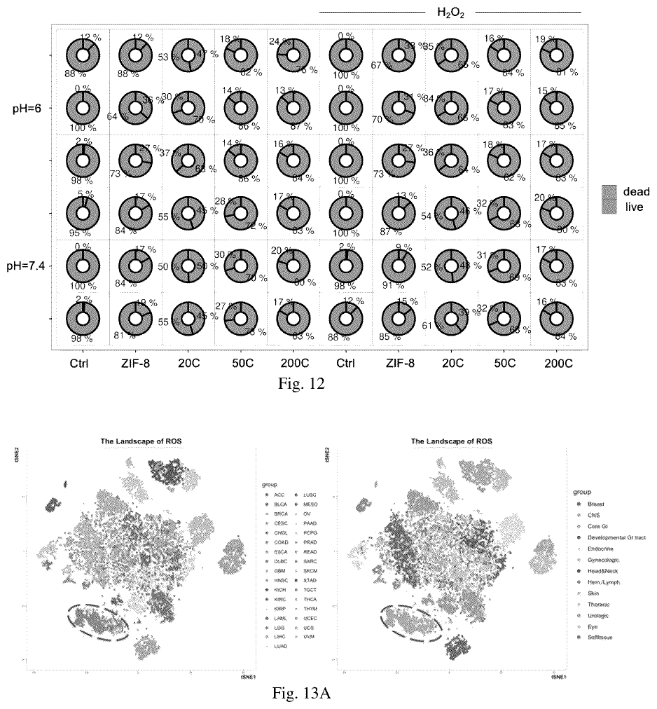

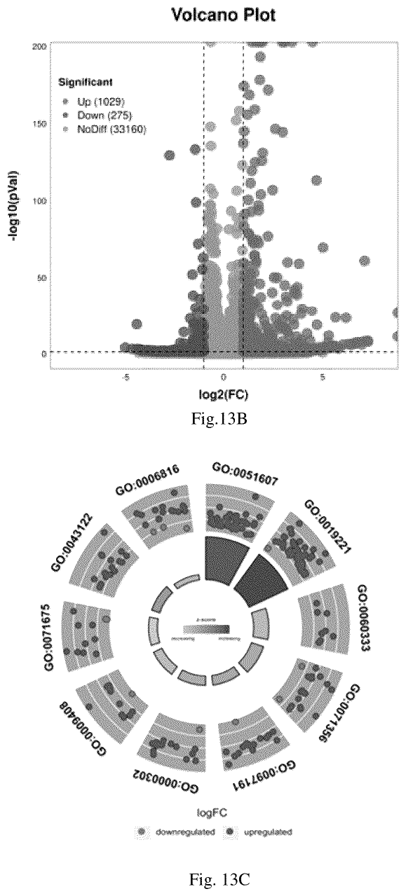

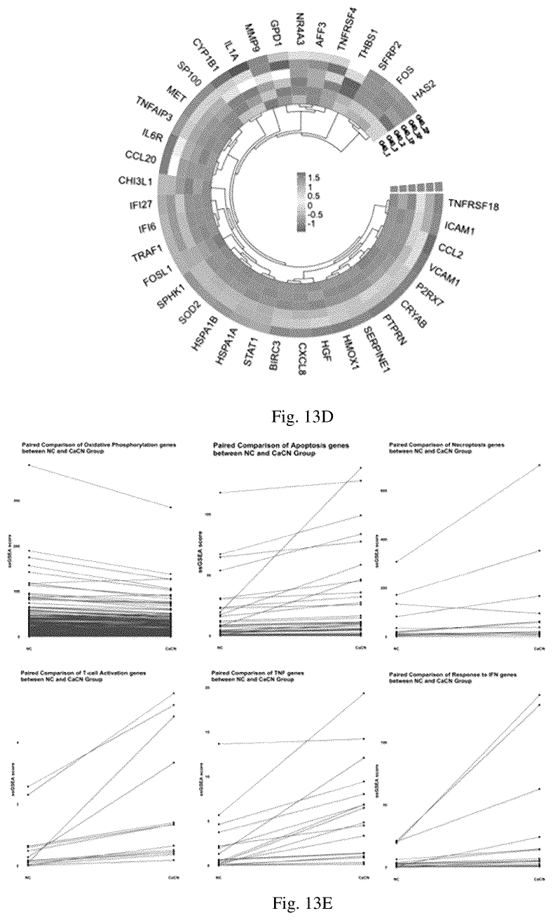

To discriminate tumor samples according to ROS status, an unsupervised consensus cluster (a well-established machine learning method) was used to classify 10000 samples across 33 cancer types based on the transcriptomic expression of the 35 ROSCGs. PCA analysis revealed that the ROS metabolic ability of glioblastoma patients is significantly different from that of other cancer patients; the metabolic capacity of central nervous system tumors is also strikingly unlike other system tumors (FIG. 13A). To further elucidate other therapeutic mechanisms caused by Ca-SAzymes, the transcriptome analysis was performed to reveal the messenger RNA (mRNA) variations of GBM21 cells after different treatments. There are 1304 expressed genes significantly different between Ca-SAzymes group and control group, involving 1029 upregulated and 275 downregulated mRNAs (FIG. 13B). Based on this discrepancy, gene ontology (GO) analysis and KEGG analysis were performed to elaborate the biological function of altered mRNAs and the corresponding influence pathways (FIG. 13C), demonstrating that the immune response was associated with the therapeutic mechanisms of the Ca-SAzymes, including tumor necrosis factor (TNF) signaling pathway and interferon-gamma-mediated (IFN-γ) signaling pathway.

Inspired by GBM microenvironment immune modulation verified by TNF and IFN signaling pathway, the related genes enriched in TNF and IFN-γ signaling pathway were further investigated (FIGS. 13D, 13E). It can be found that the main genes including TNFRSF18, ICAM1, CCL2, and VCAM1, a series of inflammatory genes have distinct expression differences in the Ca-SAzymes group compared to the control group. In addition to high-throughput sequencing, we applied mRNA to identify the differential expression of TNF and IFN (FIG. 13F), and the results were consistent with the former. Considering that the changes in inflammatory factors are related to the regulation of the fate of macrophages, we immediately verified the changes in the immune microenvironment after administration of the polarization of situational cells. As expected, with the increase of Ca-SAzymes concentration, macrophages gradually polarize towards the inflammatory direction in RNA (FIG. 13G) and protein (FIG. 13H) expression, which could be attributed to inhibiting the malignant progression of GBM. In FIGS. 13A-13G, values are presented as means±s.d. (experiments were performed in quadruplicates). *P<0.05, **P<0.01, ***P<0.001.

### Example 6

### Vivo Tumor Catalytic Therapy of Ca-SAzymes

The in vivo activity of Ca-SAzymes prepared according to Example 1 was tested in this example.

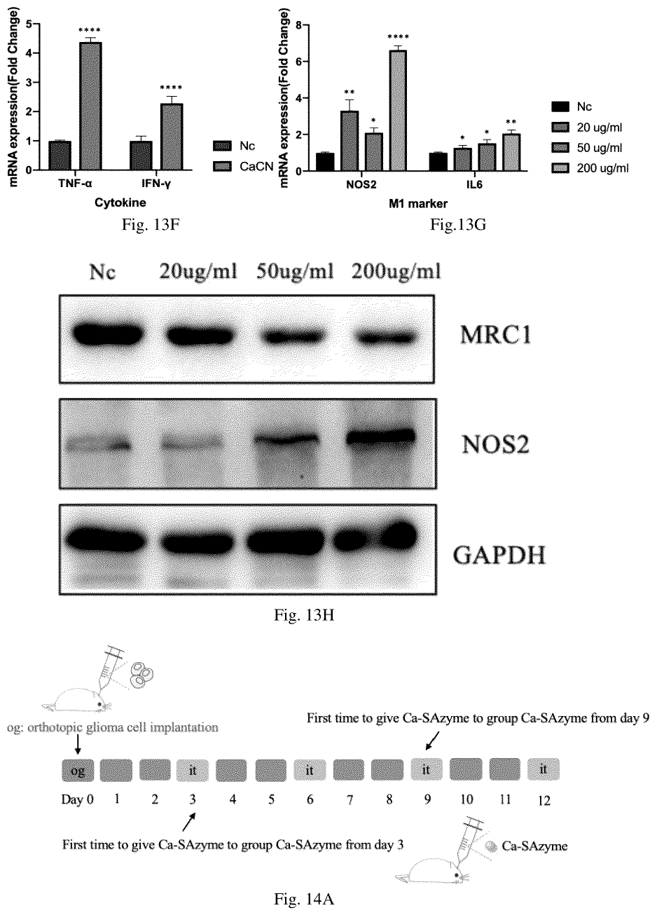

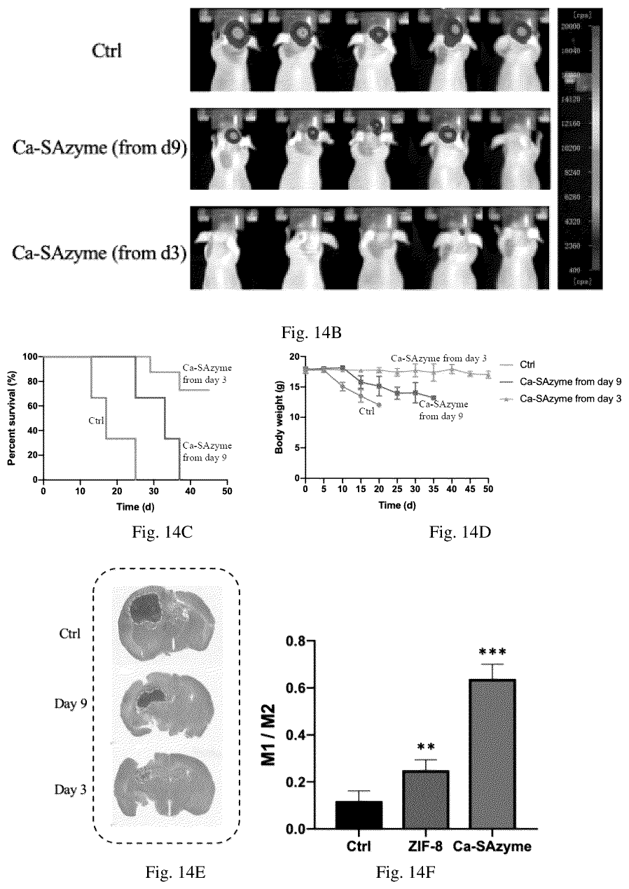

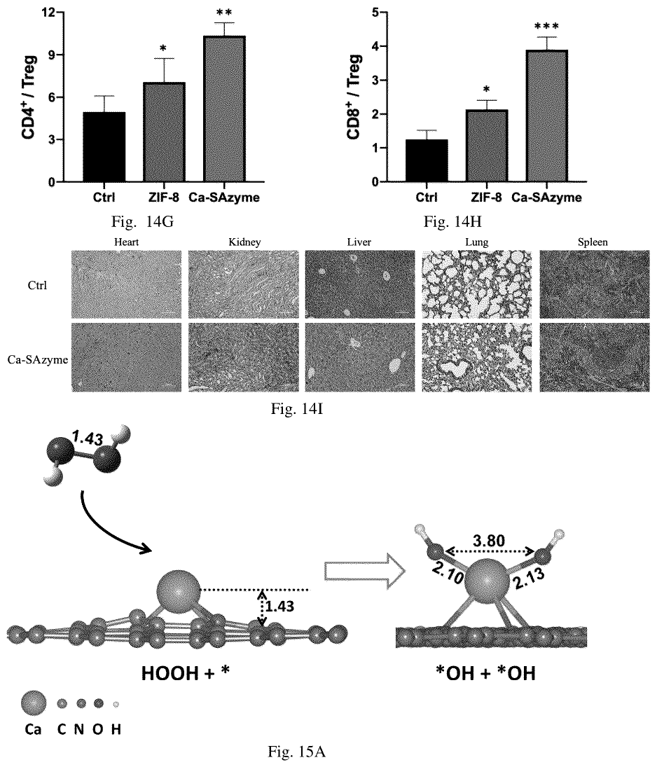

Encouraged by the results from the in vitro and microenvironment studies, we performed in vivo experiments to explore the catalytic therapy ability of Ca-SAzymes to suppress glioblastoma growth in an intracranial glioblastoma model in situ. FIG. 14A presents a schematic depiction of the design and proposed mechanism of the action of Ca-SAzymes. Distinguished from the control group that only received PBS with ZIF-8, the experimental group was divided into a Ca-SAzymes (from day 3) group administered from day 3 and a Ca-SAzymes (from day 9) group administered from day 9. The results showed significant glioblastoma growth inhibition in mice treated with Ca-SAzymes compared with controls after 21 days of regular dosing (FIG. 14B). From the results of animal imaging, the earlier the time of administration, the better the prognosis of mice might be, which reemphasizes the importance of early administration of clinical transformational drugs. Corresponding to previous results, mice treated with Ca-SAzymes (from day 3) achieved an 80% survival rate after 50 days and Ca-SAzymes (from day 9) could extend survival to a certain extent (FIG. 14C). During 50 days of the therapeutic period, the body weights for Ca-SAzymes (from day 3) did not show obvious change, implying no significant toxicity for Ca-SAzymes (FIG. 14D). Comparatively, the untreated mice lost weight dramatically due to cachexia depletion of the glioblastoma, which further confirmed the successful catalytic therapy of Ca-SAzymes for glioblastoma.

The immune microenvironment of glioblastoma has always been an important prognostic link of glioblastoma, especially since the pro-inflammatory immune environment changes may inhibit tumor progression. To get deep insight into TME regulation induced by Ca-SAzymes, the glioblastoma-bearing mice (C57BL/6) with various treatments were euthanized and the corresponding tumor tissues were analyzed. As expected, compared with control groups, an obvious increase in the M1/M2 ratio was observed for the Ca-SAzymes group (FIG. 14E), indicating an improved glioblastoma immune microenvironment. Over and above, significantly increased rations of CD⁴⁺ T to Treg cells and CD⁸⁺ T to Treg cells were observed within the Ca-SAzymes group, which indicates the TME reprogramming (FIGS. 14F-14I). In addition to programmed death, the in vivo Reprogramming of peritumoral immunity caused by Ca-SAzymes might be another cause of solid tumor shrinkage.

We further assessed the biosafety of Ca-SAzymes by pathological analysis of the main organs of healthy mice after intravenous or intratumorally injection of Ca-SAzymes at therapeutic doses (FIG. 14I). No abnormal organ pathological changes or body weight loss were observed in mice treated with Ca-SAzymes, indicating the safety of Ca-SAzymes treatment in vivo for anti-glioblastoma applications.

### Example 7

### Potential Catalytic Mechanism

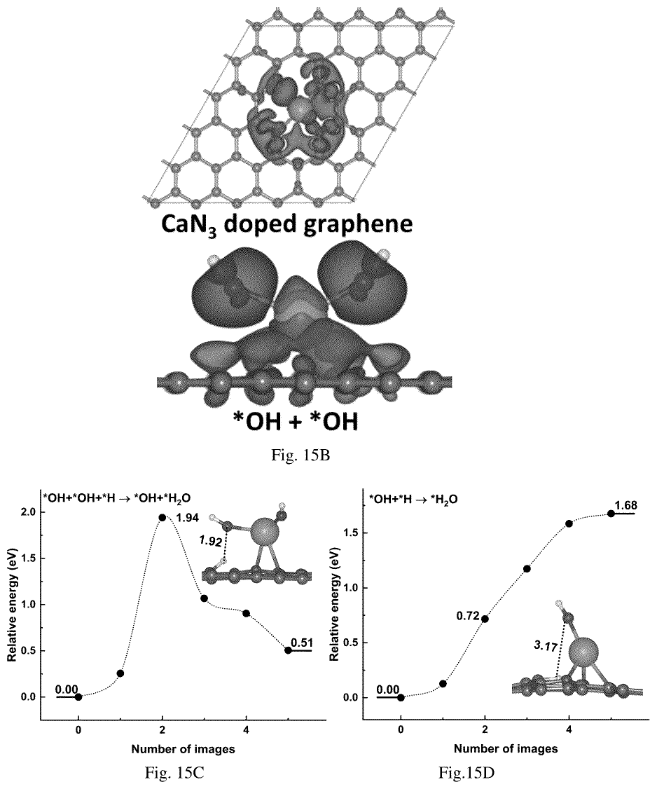

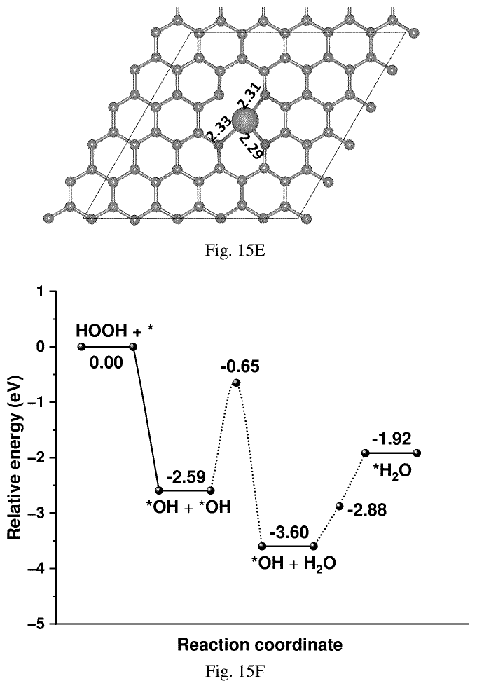

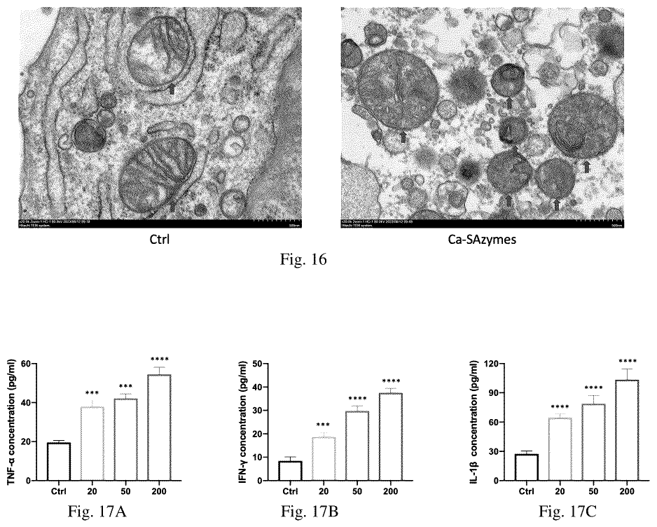

For DFT calculations, the CaN₃ doped graphene was built in a 5×5 graphene supercell (FIG. 15A and FIG. 15E). The Ca atom is bound with three N and one C atoms with bond lengths of about 2.30 Å and it is 1.43 Å above the surface. The differential charge density distribution reveals that after doping, charge migrations only exist on the CaN₃C moiety and there is no charge change for the other C atoms, demonstrating the CaN₃C moiety as the active site. As shown in FIG. 15A, before adsorption, the distance of the O—O bond in the free HOOH molecule is 1.43 Å. Owing to the large coulomb exclusion between the negatively charged O atoms with a net charge of about −1.20 |e| (FIG. 15B), the HOOH is simultaneously dissociated to *OH+*OH during the adsorption process, where the distance between two O atoms is elongated to 3.80 Å. However, for the subsequent hydrogenation reaction *OH+*OH+*H→*OH+*H₂O (FIG. 15C), the long-distance (D_{O—H}=1.92 Å) between the O and H atoms at TS results in the high energy barrier of 1.94 eV with an endothermic energy of 0.51 eV. After the H₂O molecule is released from the surface, the *OH is hydrogenated to *H₂O. Although this reaction needs to overcome a smaller energy barrier of 0.72 eV, the endothermic energy of 1.68 |e| is much higher, also caused by the long distance of D_{O—H}=3.17 Å (FIG. 15D). The calculation results prove that HOOH can be easily dissociated because of the high activity of Ca atom. Besides, the large size of Ca atom leads to space resistance for the high energy barrier and endothermic energy. This will protect *OH from subsequent hydrogenations (FIG. 15F). Therefore, there will be lots of OH released from the surface of CaN₃ doped graphene.

### CONCLUSION

This application has developed a microenvironment responsive single-atom calcium nanozyme for glioblastoma treatment that induces tumor cell apoptosis via elevating ROS and reversing glioblastoma immune microenvironment. The Ca-SAzymes exhibit the higher affinity and better activation ability towards H₂O₂, confirmed by experiments and DFT calculations. By inducing the breakdown of H₂O₂ into ROS, Ca-SAzymes achieve effective killing of tumor cells in vitro. Moreover, Ca-SAzymes significantly prolong the survival of mice in glioblastoma-bearing mice model and reversed glioblastoma immune microenvironment without adverse effects or toxicity. Collectively, this application provides an unprecedented insight into the catalytic treatment of glioblastoma and the reversal of the immune microenvironment.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable subcombination.

All references specifically cited herein are hereby incorporated by reference in their entireties. However, the citation or incorporation of such a reference is not necessarily an admission as to its appropriateness, citability, and/or availability as prior art to/against the present invention.

What is claimed is:

1. A single atom nanozyme comprising an alkaline earth metal single atom loaded on a carbon material, wherein the alkaline earth metal is selected from the group consisting of calcium, magnesium, barium, and a combination thereof, wherein the carbon material is nitrogen-doped and selected from the group consisting of zeolitic imidazolate framework (ZIF), carbon fiber, carbon nanotube, graphene, carbon black, reduced graphene oxide, and a combination thereof.

2. The single atom nanozyme of claim 1, wherein the alkaline earth metal is calcium, which mimic the active sites of natural metalloproteases, wherein the carbon material is zeolitic imidazolate framework selected from ZIF-8, ZIF-67, and a combination thereof, and wherein the calcium metal atoms in the nanozyme are in cationic states.

3. The single atom nanozyme of claim 1, having a particle size ranging from about 50 nm to about 100 nm.

4. A method for preparing the single atom nanozyme according to claim 1, comprising the steps of:
(A) providing a carbon material selected from the group consisting of zeolitic imidazolate framework (ZIF), carbon fiber, carbon nanotube, graphene, carbon black, reduced graphene oxide, and a combination thereof, and
(B) mixing the nitrogen-doped carbon material with an alkaline earth metal source comprising an alkaline earth metal selected from the group consisting of calcium, magnesium, barium, and a combination thereof and stirring at room temperature to provide a precursor mixture;
(C) subjecting the precursor mixture to a pyrolysis process to provide the single atom nanozymes.

5. The method of claim 4, wherein the nitrogen-doped carbon material is the zeolitic imidazolate framework, which is prepared by
(1) providing a first mixture comprising a Zn source;
(2) providing a second mixture comprising 2-methylimidazole; and
(3) mixing the first mixture and the second mixture by stirring to provide particles of the zeolitic imidazolate framework.

6. The method of claim 4, wherein the pyrolysis process is performed by heating the precursor mixture to a temperature of about 900° C. to about 1010° C. at a rate ranging from greater than 0 to about 10° C./min in an inert atmosphere.

7. The method of claim 4, further comprising centrifuging and drying the precursor mixture in vacuum at 65° C. overnight prior to the pyrolysis process.

8. The method of claim 4, wherein the zeolitic imidazolate framework is ZIF-8 or ZIF-67, and the alkaline earth metal source comprises calcium.

9. The method of claim 8, wherein the organic ligands of ZIF-8 or ZIF-67 are transformed to an N-doped carbon structure and the calcium atoms are trapped by the N-doped carbon structure during the pyrolysis process.

10. The method of claim 6, wherein the pyrolysis process is performed under Ar atmosphere and Ar gas is supplied at a rate of about 10 mL/min.

11. A method of tumor catalytic therapy comprising administrating a nanozyme comprising a single-atom alkaline earth metal loaded on a nitrogen-doped carbon material to a subject in need thereof, wherein the alkaline earth metal is selected from the group consisting of calcium, magnesium, barium, and a combination thereof, wherein the carbon material is selected from the group consisting of zeolitic imidazolate framework (ZIF), carbon fiber, carbon nanotube, graphene, carbon black, reduced graphene oxide, and a combination thereof.

12. The method of claim 11, wherein the tumor is a refractory microenvironmental immunosuppressive tumor selected from the group consisting of bladder cancer, bone tumor, breast cancer, and other brain cancer, a combination thereof.

13. The method of claim 12, wherein the refractory microenvironmental immunosuppressive tumor is the brain cancer selected from the group consisting of glioblastoma, acoustic neuroma, astrocytoma, chordoma, CNS lymphoma, craniopharyngioma, brain stem glioma, ependymoma, mixed glioma, optic nerve glioma, subependymoma, medulloblastoma, meningioma, metastatic brain tumors, oligodendroglioma, pituitary tumors, Primitive Neuroectodermal (PNET), Other Brain-Related Conditions, schwannoma, and a combination thereof.

14. The method of claim 11, wherein the nanozyme is administrated at a therapeutically effective amount of from about 2 mg to about 3 mg per kg body weight.

15. The method of claim 11, wherein the loading percentage of the alkaline earth metal in the nanozyme ranges from greater than 0 to about 2.0 wt %.

16. The method of claim 11, wherein the nanozyme has comparable peroxidase-like catalytic (POD) activity and kinetics to natural enzymes.

17. The method of claim 11, wherein the nanozyme acts as an immunoadjuvant to awaken innate immunity by stimulating the tumor to produce cytokine.

18. The method of claim 11, wherein the nanozyme induces tumor cell apoptosis via elevating ROS and reversing glioblastoma immune microenvironment.

19. The method of claim 11, wherein the nanozyme acts as an immunoadjuvant to awaken innate immunity by stimulating the tumor to produce cytokine, which promotes polarization of tumor-associated macrophages to reverse the immunosuppressive microenvironment.

20. The method of claim 11, wherein the nanozyme, as an exotic Ca supplier, causes mitochondrial Ca overload, which further amplifies oxidative stress in tumor.


SEQUENCE LISTING (10 sequences)

SEQ ID NO: 1 - DNA, 23 bp, synthetic construct
  source 1..23: mol_type = other DNA
tcggtaactgacttgaatgtcca

SEQ ID NO: 2 - DNA, 21 bp, synthetic construct
  source 1..21: mol_type = other DNA
tcgcttccctgttttagctgc

SEQ ID NO: 3 - DNA, 20 bp, synthetic construct
  source 1..20: mol_type = other DNA
tcccacctgaccttgtgctt

SEQ ID NO: 4 - DNA, 21 bp, synthetic construct
  source 1..21: mol_type = other DNA
cagggcgtaccactttagctc

SEQ ID NO: 5 - DNA, 23 bp, synthetic construct
  source 1..23: mol_type = other DNA
aacaaattcggtacatcctcgac

SEQ ID NO: 6 - DNA, 21 bp, synthetic construct
  source 1..21: mol_type = other DNA
attttcaccaggcaagtctcc

SEQ ID NO: 7 - DNA, 21 bp, synthetic construct
  source 1..21: mol_type = other DNA
tcgaaccccgagtgacaagcc

SEQ ID NO: 8 - DNA, 21 bp, synthetic construct
  source 1..21: mol_type = other DNA
gttcttatactcgggccacac

SEQ ID NO: 9 - DNA, 20 bp, synthetic construct
  source 1..20: mol_type = other DNA
acgaccactttgtcaagctc

SEQ ID NO: 10 - DNA, 23 bp, synthetic construct
  source 1..23: mol_type = other DNA
ctgttgctgtagccaaattcgtt